(19) United States
(12) Patent Application Publication
(10) Pub. No.: US 2007/0010548 A1
(43) Pub. Date: Jan. 11, 2007

(54) Compounds having inhibitive activity of phosphatidylinositol 3-kinase and methods of use thereof

(75) Inventors: Beth E. Drees, Salt Lake City, UT (US); Leena Chakravarty, Sandy, UT (US); Glenn D. Prestwich, Salt Lake City, UT (US); Gyorgy Dorman, Budapest (HU); Mariann Kavecz, Varpalota (HU); Andras Lukacs, Budapest (HU); Laszlo Urge, Budapest (HU); Ferenc Darvas, Budapest (HU); Piotr W. Rzepecki, Salt Lake City, UT (US); Colin G. Ferguson, Salt Lake City, UT (US)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: ZENTARIS GmbH, Frankfurt (DE)

(21) Appl. No.: 10/560,591

(22) PCT Filed: Jun. 14, 2004
(86) PCT No.: PCT/US04/18752

§ 371 Date: Aug. 24, 2006

Related U.S. Application Data
(60) Provisional application No. 60/478,221, filed on Jun. 13, 2003.

(51) Int. Cl. A61K 31/4745; C07D 471/02; C12Q 1/48

(52) U.S. Cl. 514/292; 546/82; 546/119; 514/303; 435/15

(57) ABSTRACT

Compounds inhibiting phosphatidylinositol 3-kinase (PI 3-K) activities and methods of preparing and using thereof in treating diseases are disclosed. Compounds inhibiting PI 3-K activity and methods of using PI 3-K inhibitory compounds to inhibit cancer cell grwoth or to treat disorders of immunity and inflammation, in which PI 3-K plays a role in leukocyte function are also provided.

### BACKGROUND OF THE INVENTION

1. Field of the Invention

The present invention relates generally to phosphatidylinositol 3-kinase (PI 3-K) enzymes, and more particularly to inhibitors of PI 3-K activity and to methods of using such materials.

2. Related Art

The behavior of all cellular communications is governed by signaling systems which translate external signals such as hormones, neurotransmitters, and growth factors into intracellular second messengers. Phosphoinositide polyphosphates (PIPn) are key lipid second messengers in cellular signaling (Martin, Ann. Rev. Cell Dev. Biol., 14:231-2614 (1998)). Because their activity is determined by their phosphorylation state, the enzymes that modify these lipids are central to the correct execution of signaling events (Leslie, et al., Chem Rev, 101:2365-80. (2001)). Disruptions in these processes are common to many disease states, including cancer, diabetes, inflammation, and cardiovascular disease.

The production of the phosphoinositide polyphosphate PI(3,4,5)P₃ or PIP₃ by phosphatidylinositol 3-kinase (PI 3-K) is important in pathways governing cell proliferation, differentiation, apoptosis, and migration. Alterations which affect correct regulation of PIP₃ levels and the levels of their lipid products are associated with a variety of cancer types (Phillips et al., Cancer 83:41-47. (1998), Shayesteh, et al., Nat Genet, 21:99-102. (1999), Ma, et al., Oncogene, 19:2739-44. (2000)). Mutations which affect the regulation of PI 3-K signaling contribute to abnormal proliferation and tumorigenesis (Li, et al., Science, 275:1943-7. (1997), Teng, et al., Cancer Res, 57:5221-5. (1997)) (Shayesteh, et al., Nat Genet, 21:99-102. (1999), Ma, et al., Oncogene, 19:2739-44. (2000)).

When activated by tyrosine kinase receptors in response to growth factor stimulation, PI 3-K catalyzes the formation of PIP₃. By increasing cellular levels of PIP₃, PI 3-K induces the formation of defined molecular complexes that act in signal transduction pathways. Most notably, PI 3-K activity suppresses apoptosis and promotes cell survival through activation of its downstream target, PKB/Akt (Franke, et al., Cell, 81:727-36. (1995), Datta, et al., J Biol Chem, 271:30835-9. (1996)). The lipid phosphatases PTEN and SHIP are two enzymes that both act to decrease the cellular levels of PIP₃ by conversion either to PI(4,5)P₂ or PI(3,4)P₂.

Presently, the PI 3-kinase enzyme family has been divided into three classes based on their substrate specificities. Class I PI 3-Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI 3-Ks phosphorylate PI and phosphatidylinositol-4-phosphate, whereas Class III PI 3-Ks can only phosphorylate PI. Eight separate isoforms of PI 3-K have been characterized in humans.

The initial purification and molecular cloning of PI 3-kinase revealed that it was a heterodimer consisting of p85 and p110 subunits (Otsu et al., Cell, 65:91-104 (1991); Hiles et al., Cell, 70:419-29 (1992)). Since then, four distinct Class I PI 3-Ks have been identified, designated PI 3-K alpha, beta, delta, and gamma, each consisting of a distinct 110 kDa catalytic subunit and a regulatory subunit. More specifically, three of the catalytic subunits, i.e., p110 alpha, p110 beta and p110 delta, each interact with the same regulatory subunit, p85; whereas p110 gamma interacts with a distinct regulatory subunit, p101. In each of the PI 3-Kinase alpha, beta, and delta subtypes, the p85 subunit acts to localize PI 3-kinase to the plasma membrane by the interaction of its SH2 domain with phosphorylated tyrosine residues (present in an appropriate sequence context) in target proteins Two isoforms of p85 have been identified, p85 alpha, which is ubiquitously expressed, and p85 beta, which is primarily found in the brain and lymphoid tissues. Association of the p85 subunit to the PI 3-kinase p110 alpha, beta, or delta catalytic subunits appears to be required for the catalytic activity and stability of these enzymes. In addition, the binding of Ras proteins also upregulates PI 3-kinase activity. Though a wealth of information has been accumulated in recent past on the cellular functions of PI 3-kinases in general, in particular for PI 3-K alpha and PI 3-K gamma, the roles played by the individual isoforms are have yet to be clearly defined. Details concerning the p110 isoform also can be found in U.S. Pat. Nos. 5,858,753; 5,822,910; and 5,985,589.

Specific inhibitors against individual members of a family of enzymes provide invaluable tools for deciphering the functions of each enzyme. Experimental usage of PI 3-K inhibitors has contributed to the current understanding of the role of PI 3-K activity in normal function and in disease. The major pharmacological tools used in this capacity are wortmannin (Powis, et al., Cancer Res, 54:2419-23. (199), and bioflavenoid compounds, including quercetin (Matter et al., Biochem. Biophys. Res. Commun. 186:624-631. (1992)) and LY294002 (Vlahos, et al., J Biol Chem, 269:5241-8. (1994)). The concentrations of wortmannin needed to inhibit PI 3-Ks range from 1-100 nM, and inhibition occurs via covalent modification of the catalytic site (Wymann et al., Mol. Cell. Biol. 16:1722-1733. (1996)). The bioflavenoid quercetin effectively inhibits PI 3-K with an IC₅₀ of 3.8 μM, but has poor selectivity, as it also shows inhibitory activity toward PI 4-kinase, and several protein kinases. LY294002 is a synthetic compound made using quercetin as a model, inhibits PI 3-K with an IC₅₀ of 100 L (Vlahos, et al., J Biol Chem, 269:5241-8. (1994)). Both quercetin and LY294002 are competitive inhibitors of the ATP binding site of PI 3-K, however, only LY294002 shows specificity for inhibition of PI 3-K and does not affect other types of kinases. Both wortmannin and LY294002 have been used extensively to characterize the biological roles of PI 3-K, however, neither shows selectivity for individual PI 3-K isoforms. Hence, the utility of these compounds in studying the roles of individual Class I PI 3-kinases is limited.

The PI 3-K inhibitors are expected to be a new type of medication useful for cell proliferation disorders, in particular as antitumor agents. As PI 3-K inhibitors, wortmannin [H. Yano et al., J. Biol. Chem., 263, 16178 (1993)] and LY294002 [J. Vlahos et al., J. Biol. Chem., 269, 5241(1994)] which is represented by the formula below, are known. However, creation of PI 3-K inhibitors having more potent cancer cell growth inhibiting activity is desired.

Because many oncogenic signaling pathways are mediated by PI 3-K, inhibitors that target PI 3-K activity may have application for the treatment of cancer. Studies using comparative genomic hybridization revealed several regions of recurrent abnormal DNA sequence copy number that may encode genes involved in the genesis or progression of ovarian cancer. One region found to be increased in copy number in approximately 40% of ovarian and other cancers contains the PIK3CA gene, which encodes the p110 alpha catalytic subunit of PI 3-K alpha This association between the PIK3CA copy number and PI 3-kinase activity makes PIK3CA a candidate oncogene because a broad range of cancer-related functions have been associated with PI 3-kinase-mediated signaling. PIK3CA is frequently increased in copy number in ovarian cancers, and increased copy number is associated with increased PIK3CA transcription, p110-alpha protein expression, and PI 3-kinase activity (Shayesteh, et al., Nature Genet. 21: 99-102, (1999)). Furthermore, treatment of ovarian cancer cell lines exhibiting increased PI 3-K activity and Akt activation with a PI 3-kinase inhibitor decreased proliferation and increased apoptosis (Shayesteh, et al., Nature Genet. 21: 99-102, (1999), Yuan et al., Oncogene 19:2324-2330. (2000)). Thus, PI 3-K alpha has an important role in ovarian cancer. In cervical cancer cell lines harboring amplified PIK3CA, the expression of the gene product was increased and was associated with high PI 3-kinase activity (Ma et al., Oncogene 19: 2739-2744, (2000)). Thus, increased expression of PI 3-kinase alpha in cervical cancer may promote cell proliferation and reduce apoptosis. In addition, mutation of the lipid phosphatase and tumor suppressor PTEN, a 3′ phosphatase that breaks down PIP₃, is one of the most common cancer-associated mutations, and is particularly associated with glioblastoma, prostate, endometrial, and breast cancers (Li et al., Science 275:1943-1947 (1997), Teng et al., Cancer Res. 57:5221-5225. (1997), Ali et al., J. National Cancer Institute, 91:1922-1932. (1999), Simpson and Parsons, Exp. Cell Res. 264:29-41 (2002)). PI 3-K activity suppresses apoptosis and promotes cell survival largely through activation of its downstream target, PKB/Akt (Franke et al. Cell 81:727-736. (1995), Dattaet al., J Biol Chem 271:30835-30839 (1996)). Akt activation and amplification is present in many cancers (Testa and Bellicosa, Proc. Natl. Acad. Sci. USA 98:10983-10985. (2002)).

Treatment with PI 3-K inhibitors has been shown to block proliferation of several cancer cell lines, and to be an effective treatment for tumor xenograft models in addition to ovarian carcinoma. Akt is activated in a majority of non-small cell lung cancer cell lines, and treatment with PI 3-K inhibitors causes proliferative arrest in these cells (Brognard et al., Cancer Res. 60:6353-6358. (2000), Lee et al., J. Biol. Chem. electronic publication, (2003)). The PI 3-K/Akt pathway is also constitutively activated in a majority of human pancreatic cancer cell lines, and treatment with PI 3-K inhibitors induced apoptosis in these cell lines. Decreased tumor growth and metastasis was also observed upon treatment with PI 3-K inhibitors in a xenograft model of pancreatic cancer (Perugini et al., J. Surg. Res. 90:39-44 (2000), Bondar et al., Mol. Cancer Ther. 1:989-997 (2002)). Treatment with LY204002 induced growth arrest and apoptosis in PTEN-deficient human malignant glioma cells (Shingu et al., J. Neurosurg. 98:154-161. (2003)). LY294002 produces growth arrest in human colon cancer cell lines and suppression of tumor growth in colon carcinoma xenografts in mice (Semba et al., Clin Cancer Res. 8:1957-1963. (2002)). Inhibitors of PI 3-K inhibit in vitro anchorage-independent growth and in vivo metastasis of liver cancer cells (Nakanishi et al., Cancer Res. 62:2971-2975. (2002)). Treatment of Burkitt's lymphoma cells with LY294002 induces apoptosis (Brennan et al., Oncogene 21:1263-1271. (2002)). LY294002 also has been shown to induce apoptosis in multi-drug resistant cells (Nicholson et al., Cancer Lett. 190:31-36. (2003)). Thus, PI 3-K inhibitors maybe suitable therapeutics agents for many tumors exhibiting activated or increased levels of PI 3-K or PKB/Akt as well as for tumors which are PTEN-deficient.

Several studies have demonstrated that agents which target the PI 3-K pathway can enhance the effects of standard chemotherapeutic agents in a variety of cancer types. Thus, PI 3-K inhibitors may have value as novel adjuvant therapies for certain cancers. PI 3-K inhibitors induce apoptosis in pancreatic carcinoma cells exhibiting constitutive phosphorylation and activation of AKT, and suboptimal doses produce additive inhibition of tumor growth when combined with a suboptimal dose of gemcitabine (Ng, et al., Cancer Res, 60:5451-5. (2000, Bondar, et al., Mol Cancer Ther, 1:989-97. (2002)). Inhibition of PI 3-K also increases the responsiveness of pancreatic carcinoma cells to the non-steroidal anti-inflammatory agent (NSAID) sulindac (Yip-Schneider, et al., J Gastrointest Surg, 7:354-63. (2003)). In a mouse xenograft model of pancreatic cancer, a combination of wortmannin with gemcitabine also showed increased efficacy in induction of tumor apoptosis relative to treatment with each agent alone (Ng, et al., Clin Cancer Res, 7:3269-75. (2001)). In an athymic mouse xenograft model of ovarian cancer, combined treatment with LY294002 and paclitaxal results in increased efficacy of paclitaxal-induced apoptosis of tumor cells, and allows the use of decreased levels of LY294002, resulting in less dermatological toxicity (Hu, et al., Cancer Res, 62:1087-92. (2002)). HL60 human leukemia cells show sensitization to cytotoxic drug treatment and Fas-induced apoptosis when treated with PI 3-K inhibitors, suggesting a role for PI 3-K inhibition in treating drug resistant acute myeloid leukemia (O'Gorman, et al., Leukemia, 14:602-11. (2000, O'Gorman, et al., Leuk Res, 25:801-11. (2001)). Inhibition of PI 3-K enhances the apoptotic effects of sodium butyrate, gemcitabine, and 5-fluoruracil in aggressive colon cancer cell lines (Wang, et al., Clin Cancer Res, 8:1940-7. (2002)). LY294002 potentiates apoptosis induced by doxorubicin, trastumazab, paclitaxal, tamoxifen, and etoposide in breast cancer cell lines exhibiting PTEN mutations or erbB2 overexpression (Clark, et al., Mol Cancer Ther, 1:707-17. (2002)). Inhibition of PI 3-K potentiates the effect of etoposide to induce apoptosis in small cell lung cancer cells (Krystal, et al., Mol Cancer Ther, 1:913-22. (2002)).

In addition to enhancing the effects of chemotherapeutic agents for cancer treatment, PI 3-K inhibitors also may enhance tumor response to radiation treatment. Inhibitors of PI 3-K revert radioresistance in breast cancer cells transfected with constitutively active H-ras (Liang, et al., Mol Cancer Ther, 2:353-60. (2003)), and PI 3-K inhibitors enhance radiation-induced apoptosis and cytotoxicity in tumor vascular endothetial cells (Edwards, et al., Cancer Res, 62:4671-7. (2002)). Thus, PI 3-K inhibitors could be used to enhance response to radiotherapy, both in tumor cells and in tumor vasculature.

U.S. Pat. No. 6,403,588 discloses imidazopyridine derivatives having excellent PI 3-K inhibiting activity and cancer cell growth inhibiting activity. U.S. Pat. No. 5,518,277 discloses compounds that inhibit PI 3-K delta activity, including compounds that selectively inhibit PI 3-K delta activity. However, all of these compounds have a structure different from those of the present invention.

### SUMMARY OF THE INVENTION

It has been recognized that it would be advantageous to develop inhibitors of PI 3-K polypeptides. In particular, inhibitors of PI 3-K are desirable for exploring the roles of PI 3-K isozymes and for development of pharmaceuticals to modulate the activity of the isozymes.

One embodiment of the present invention is to provide a compound which is useful as a phosphatidylinositol 3-kinase (PI 3-K) inhibitor having a general structure represented by Formula I, Formula II, or Formula III;

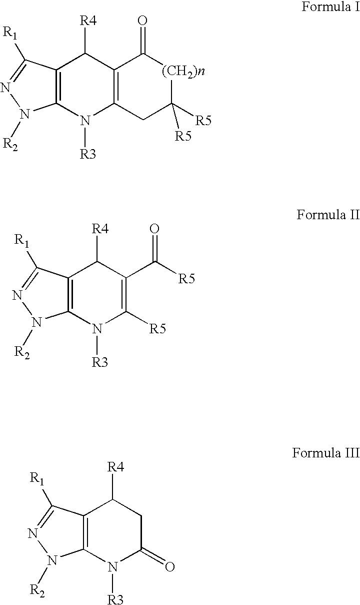

wherein n can be an integer selected from 0 to 2.

In one aspect, R₁ and R₂ can be each independently a moiety selected from the group consisting of hydrogen, alkyl, alkenyl, aryl, hetaryl, aralkyl, hetaralkyl, alkyl substituted with at least one substituent, aryl substituted with at least one substituent, hetaryl substituted with at least one substituent, aralkyl substituted with at least one substituent, and hetaralkyl substituted with at least one subsituent. In another aspect, R₃ can be a moiety selected from the group consisting of hydrogen, alkyl, alkenyl, aralkyl, alkyl substituted with at least one substituent, aralkyl substituted with at least one substituent, CO—R₅, SO₂—R₅; CO—O—R₅, CO—N—R₄, and R₅ In an additional aspect, R₄ and R₅ can be each independently a moiety selected from the group consisting of hydrogen, alkyl alkenyl, cycloalkyl, aralkyl, aryl, alkyl substituted with at least one substituent, cycloalkyl substituted with a substituent, aryl substituted with at least one substituent, and aralkyl substituted with at least one substituent.

One embodiment of the present invention is a compound which is useful as a phosphatidylinositol 3-kinase (PI 3-K) inhibitor having a general structural represented by Formula I, II, or III wherein said alkyl, cycloalkyl, or aralkyl is a C₁₋₁₅ alkyl, C₃₋₈ cycloalkyl, C₂₋₁₈ alkenyl or aralkyl group is substituted by 1 to 5 substituents selected from the group consisting of nitro, hydroxy, cyano, carbamoyl, mono- or di-C₁₋₄ alkyl-carbamoyl, carboxy, C₁₋₄ alkoxy-carbonyl, sulfo, halogen, C₁₋₄ alkoxy, phenoxy, halophenoxy, C₁₋₄ alkylthio, mercapto, phenylthio, pyridylthio, C₁₋₄ alkylsulfinyl, C₁₋₄ alkylsulfonyl, amino, C₁₋₃ alkanoylamino, mono- or di-C₁₋₄ alkylamino, 4- to 6-membered cyclic amino, C₁₋₃ alkanoyl, benzoyl and 5 to 10 membered heterocyclic groups.

Another embodiment of the present invention is a compound which is useful as a phosphatidylinositol 3-kinase (PI 3-K) inhibitor having a general structural represented by Formula I, II or III wherein said alkyl is a straight or branched hydrocarbon chain having 1 to 15 carbon atoms, said aryl is an aromatic cyclic hydrocarbon group having 6 to 14 carbon atoms, said hetaryl is a 5- or 6-membered monocyclic heterocyclic group containing 1 to 4 hetero-atoms selected from oxygen, sulfur and nitrogen or a fused bicyclic heterocyclic group containing 1 to 6 hetero-atoms selected from oxygen, sulfur and nitrogen, said substituted aryl is a C₆₋₁₄ aryl group which is substituted by 1 to 4 substituents selected from the group consisting of halogen, C₁₋₄ alkyl, C₁₋₄ haloalkyl, C₁₋₄ haloalkoxy, C₁₋₄ alkoxy, C₁₋₄ alkylthio, hydroxy, carboxy, cyano, nitro, ammo, mono- or di-C₁₋₄ alkylamino, formyl, mercapto, C₁₋₄ alkyl-carbonyl, C₁₋₄ alkoxy-carbonyl, sulfo, C₁₋₄ alkylsulfonyl, carbamoyl, mono- or di-C₁4 alkyl-carbamoyl, oxo and thioxo; and said substituted hetaryl is a hetaryl which is substituted by 1 to 4 substituents selected from the group consisting of halogen, C₁₋₄ alkyl, C₁₋₄ haloalkyl, C₁₋₄ haloalkoxy, C₁₋₄ alkoxy, C₁₋₄ akylthio, hydroxy, carboxy, cyano, nitro, amino, mono- or di-C₁₋₄ alkylamino, formyl, mercapto, C₁₋₄ alkyl-carbonyl, C₁₋₄ alkoxy-carbonyl, sulfo, C₁₋₄ alkylsulfonyl, carbamoyl, mono- or di-C₁₋₄ alkyl-carbamoyl, oxo and thioxo groups.

Another embodiment of the present invention is a compound which is useful as a phosphatidylinositol 3-kinase (PI 3-K) inhibitor having a general structural represented by Formula I, II or III wherein R₁ and R₂ are each independently a member selected from the group consisting of C₁₋₆ alkyl, phenyl, naphthyl, hetaryl substituted C₁₋₆ alkyl and phenyl substituted C₁₋₆ alkyl; R₃ is a member selected from the group consisting of H, C₁₋₆ alkyl, aralkyl substituted C₁₋₆ alkyl, aralkyl groups, CO—R₅, or SO₂—R₅; CO—O—R₅, CO—N—R₄, and R₅; and R₄ and R₅ can be a member selected from the group consisting of H, C₁₋₆ alkyl, substituted C₁₋₄ alkyl, cycloalkyl and aralkyl groups.

Another embodiment of the present invention is a compound which is useful as a phosphatidylinositol 3-kinase (PI 3-K) inhibitor having a general structural represented by Formula I, II or III wherein n is 1; R₁ is a member selected from the group consisting of straight chain C₁₋₆ alkyl, branched chain C₁₋₆ alkyl and phenyl groups; R₂ is a member selected from the group consisting of phenyl, C₁₋₆ alkylphenyl, C₁₋₆ dialkylphenyl, C₁₋₆ alkoxyphenyl, halophenyl, dihalophenyl and nitrophenyl groups; R₃ is a member selected from hydrogen, straight chain C₁₋₆ alkyl and branched chain C₁₋₆ alkyl groups; R₄ is a phenyl substituted with at least one substituent selected from the group consisting of aryloxy, alkylaryloxy, haloaryloxy, straight chain C₁₋₆ alkyl, branched chain C₁₋₆ alkyl, C₁₋₆ alkoxy, C₁₋₆, haloaryl and halo-C₁₋₄ alkylaryl groups; and R₅ is a straight or branched chain C₁₋₆ alkyl group.

Preferred embodiment of the present invention is a compound which is useful as a phosphatidylinositol 3-kinase (PI 3-K) inhibitor having a general structural represented by Formula I, II or III, wherein R₁ is a phenyl or a tertbutyl group; R₂ is a member selected from the group consisting of methylphenyl, dimethylphenyl, tertbutyl, methoxyphenyl, chlorophenyl, dichlorophenyl, flurophenyl and nitrophenyl group; R₃ is hydrogen; R₄ is a phenyl substituted with at least one substituent selected from the group consisting of phenoxy, benzyloxy, halophenoxy, straight chain C₁₋₆ alkyl, branched chain C₁₋₆ alkyl, C₁₋₆ alkoxy, C₁₋₆, halophenyl and halo-C₁₋₄ alkylphenyl group; and R₅ is a straight or branched chain C₁₋₆ alkyl group.

The most preferred embodiment of the present invention is a compound which is useful as a phosphatidylinositol 3-kinase (PI 3-K) inhibitor having a general structural represented by Formula I, II or III, wherein R₁ is a phenyl or tertbutyl; R₂ is a member selected from the group consisting of methylphenyl, dimethylphenyl, tertbutyl, methoxyphenyl, chlorophenyl, dichlorophenyl, flurophenyl and nitrophenyl group; R₃ is hydrogen; R₄ is a phenyl substituted with at least one substituent selected from the group consisting of phenoxy, benzyloxy, halophenoxy, straight chain C₁₋₆ alkyl, branched chain C₁₋₆ alkyl, C₁₋₆ alkoxy, C₁₋₆, halophenyl and halo-C₁₋₄ alkylphenyl group; and R₅ is a methyl group.

The present invention further relates to novel pharmaceutical compositions, particularly to PI 3-K inhibitors and antitumor agents, comprising a compound of the present invention and a pharmaceutically acceptable carrier.

A further aspect of the present invention relates to treatment methods of disorders (especially cancers) influenced by PI 3-K, wherein an effective amount of a compound of the present invention is administered to humans or animals.

Additional features and advantages of the invention will be apparent from the detailed description which follows, taken in conjunction with the accompanying drawings, which together illustrate, by way of example, features of the invention.

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

An embodiment of the present invention relates to novel compounds which are useful as PI 3-K inhibitors and antitumor agents. The compounds of the present invention are represented by one of the following general formulas: 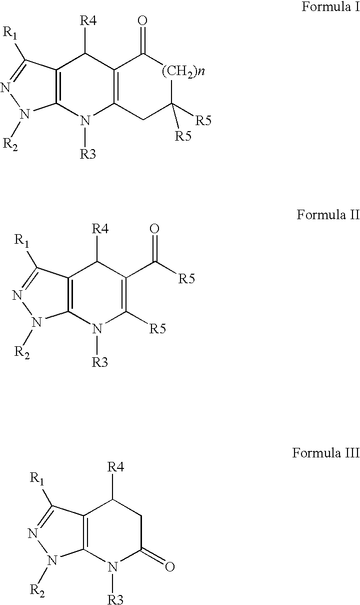
wherein n can be an integer selected from 0 to 2.

In one aspect, R₁ and R₂ can be each independently a member selected from the group consisting of hydrogen, alkyl, alkenyl, aryl, hetaryl, aralkyl, hetaralkyl, alkyl substituted with at least one substituent, aryl substituted with at least one substituent, hetaryl substituted with at least one substituent, aralkyl substituted with at least one substituent, and hetaralkyl substituted with at least one subsituent. In another aspect, R₃ can be a member selected from the group consisting of hydrogen, alkyl, alkenyl, aralkyl, alkyl substituted with at least one substituent, aralkyl substituted with at least one substituent, CO—R₅, SO₂—R₅; CO—O—R₅, CO—N—R₄, and R₅. In an additional aspect, R₄ and R₅ can be each independently a member selected from the group consisting of hydrogen, alkyl, alkenyl, cycloalkyl, aralkyl, aryl, alkyl substituted with at least one substituent, cycloalkyl substituted with at least one substituent, aryl substituted with at least one substituent, and aralkyl substituted with at least one substituent.

In accordance with the invention, the compound according to Formula I, Formula II, and/or Formula III can be substituted with various moieties, whenever any of such are used. Accordingly, the allyl can be a straight or branched chain C₁₋₁₅ alkyl. In one aspect, the cycloalkyl can be a C₃₋₈ cycloalkyl. In another aspect, the alkenyl can be a straight or branched chain C₂₋₁₈ alkenyl. In yet another aspect, the aralkyl can be a carbomonocyclic aromatic or carbobicyclic aromatic substituted with a straight or branched chain C₁₋₁₅ alkyl. In still another aspect, any of the substituents can be selected from the group consisting of nitro, hydroxy, cyano, carbamoyl, mono- or di-C₁₋₄ alkyl-carbamoyl, carboxy, C₁₋₄ alkoxy-carbonyl, sulfo, halogen, C₁₋₄ alkoxy, phenoxy, halophenoxy, C₁₋₄ alkylthio, mercapto, phenylthio, pyridylthio, C₁₋₄ alkylsulfinyl, C₁₋₄ alkylsulfonyl, amino, C₁₋₃ alkanoylamino, mono- or di-C₁₋₄ alkylamino, 4- to 6-membered cyclic amino, C₁₋₃ alkanoyl, benzoyl, and 5 to 10 membered heterocyclic groups.

In another embodiment, R₁₋₅ of Formula I, Formula II, and/or Formula III can be each individually selected from variety of moieties whenever any of such are used, where the moieties can optionally be substituted with at least one substituent. Accordingly, the aryl can be a carbomonocyclic aromatic or carbobicyclic aromatic group. In one aspect, the hetaryl can be a heteromonocyclic aromatic or heterobicyclic aromatic containing 1 to 4 hetero-atoms or 1 to 6 hetero-atoms selected from oxygen, sulfur and nitrogen. In another asepct, the aralkyl can be a carbomonocyclic aromatic or carbobicyclic aromatic substituted with a straight or branched chain C₁₋₁₅ alkyl group. In an additional aspect, the substituent can be selected from the group consisting of halogen, C₁₋₄ alkyl, C₁₋₄ haloalkyl, C₁₋₄ haloalkoxy, C₁₋₄ alkoxy, C₁₋₄ alkylthio, hydroxy, carboxy, cyano, nitro, amino, mono- or di-C₁₋₄ alkylamino, formyl, mercapto, C₁₋₄ alkyl-carbonyl, C₁₋₄ alkoxy-carbonyl, sulfo, C₁₋₄ alkylsulfonyl, carbamoyl, mono- or di-C₁₋₄ alkyl-carbamoyl, oxo, and thioxo.

In one aspect, R₁ and R₂ can be each independently a member selected from the group consisting of hydrogen, straight or branched chain C₁₋₆ alkyl, phenyl, naphthalyl, hetaryl, C₁₋₆ alkyl substituted with at least one substituted, straight or branched chain C₁₋₆ alkylphenyl, phenyl substituted with at least one substituent, and benzyl. In one aspect, R₃ can be a member selected from the group consisting of hydrogen, C₁₋₆ aLkyl, aralkyl, C₁₋₆ alkyl substituted with at least one substituent, CO—R₅, or SO₂—R₅; CO—O—R₅, CO—N—R_{4,} and R₅. In another aspect, R₄ and R₅ can be each independently a member selected from the group consisting of hydrogen, C₁₋₆ alkyl, C₁₋₆ alkyl substituted with at least one substituent, cycloalkyl, phenyl, phenyl substituted with at least one substituent, benzyl, and aralkyl groups.

In an additional embodiment, the moieties conjugated thereto can be unsubstituted or substituted with at least one substitutent. In one aspect, the alkyl can be a straight or branched chain C₁₋₁₅. In another aspect, the alkenyl can be a straight or branched chain C₂₋₁₈ alkenyl. In an additional aspect, the aryl can be a carbomonocyclic aromatic or carbobicyclic aromatic group. In yet another aspect, the cycloalkyl can be a C₃₋₈ alkyl ring. In still another aspect, the hetaryl can be a heteromonocyclic aromatic or heterobicyclic aromatic containing 1 to 6 hetero-atoms selected from the group consisting of oxygen, sulfur and nitrogen. In still another aspect, said aralkyl can be a carbomonocyclic aromatic or carbobicyclic aromatic group and substituted with a straight or branched chain C₁₋₁₅ alkyl. In a further aspect, said hetaralkyl can be a heteromonocyclic aromatic or heterobicyclic aromatic containing 1 to 4 hetero-atoms or 1 to 6 hetero-atoms selected from the group consisting of oxygen, sulfur and nitrogen and substituted with a straight or branched chain C₁₋₁₅. Furthermore, any of the substituents can be independently a member selected from the group consisting of halogen, C₁₋₄ alkyl, C₁₋₄ haloalkyl, C₁₋₄ haloalkoxy, C₁₋₄ alkoxy, C₁₋₄ alkylthio, phenoxyl, halophenoxy, phenylthio, pyridylthio, hydroxy, carboxy, cyano, nitro, amino, C₁₋₃ alkanoylamino, mono- or di-C₁₋₄ alkylamino, 4- to 6-membered cyclic amino, formyl, mercapto, C₁₋₄ alkyl-carbonyl, C₁₋₄ alkoxy-carbonyl, sulfo, C₁₋₄ alkylsulfinyl, C₁₋₄ alkylsulfonyl, C₁₋₃ alkanoyl, benzoyl, mono- or di-C₁₋₄ alkyl-carbamoyl, oxo, thioxo, 5 to 10 membered heterocyclic, and combinations thereof.

In a more specific embodiment, the moieties can be either unsubstituted or substituted with at least one substitutent. In accordance therewith, R₁ and R₂ can be each independently a member selected from the group consisting of straight or branched chain C₁₋₆ alkyl, phenyl, naphthyl, straight or branched chain C₁₋₆ alkyl substituted with at least one substituent, and phenyl substituted with at least one substituent. In one aspect, R₃ can be a member selected from hydrogen, straight or branched chain C₁₋₆ alkyl, C₁₋₆ aralkyl, and C₁₋₆ alkyl substituted with at least one substituent. In another aspect, R₄ and R₅ can be each independently a member selected from the group consisting of hydrogen, straight or branched chain C₁₋₆ aLkyl, straight or branched chain C₁₋₆ alkyl substituted with at least one substituent, cycloalkyl, phenyl, phenyl substituted with at least one substituent, C₁₋₆ aralkyl, and C₁₋₆ aralkyl substituted with at least one substituent. In yet another aspect, any of the substituents can be a member selected from the group consisting of methyl, halogen, halophenyloxy, methoxy, ethyloxy phenoxy, benzyloxy, trifluromethyl, t-butyl, and nitro.

In one aspect, R₁ can be selected from the group consisting of a straight or branched chain C₁₋₆ alkyl and phenyl. In another aspect, R₂ can be selected from the group consisting of a phenyl, C₁₋₆ alkylphenyl, C₁₋₆ dialkylphenyl, C₁₋₆ alkoxyphenyl, halophenyl, dihalophenyl, and nitrophenyl. In an additional aspect, R₃ can be selected from hydrogen and straight or branched chain C₁₋₆ alkyl. In yet another aspect, R₄ can be a phenyl substituted with at least one substituent selected from the group consisting of phenoxy, benzyloxy, halophenoxy, straight or branched chain C₁₋₆ alkyl, C₁₋₆ alkoxy, halophenyl, and halo-C₁₋₄ alkyl. In a further aspect, R₅ can be a straight or branched chain C₁₋₆ alkyl.

In another aspect, R₁ can be phenyl or t-butyl; R₂ can be a member selected from the group consisting of methylphenyl, dimethylphenyl, t-butyl, methoxyphenyl, chlorophenyl, dichlorophenyl, fluorophenyl, and nitrophenyl; R₃ can be hydrogen; R₄ can be a phenyl substituted with at least one substituent selected from the group consisting of chlorine, fluorine, phenoxy, benzyloxy, chlorophenoxy, methoxy, ethoxy, and trifluoromethyl; and R₅ can be a methyl.

The terms “substituted alkyl, cycloalkyl, alkenyl, or aralkyl” means: C₁₋₁₅ allyl, C₃₋₈ cycloalkyl, C₂₋₁₈ alkenyl or aralkyl groups which may be substituted by 1 to 5 substituents selected from the group consisting of (i) nitro, (ii) hydroxy, (iii) cyano, (iv) carbamoyl, (v) mono- or di-C₁₋₄ alkyl-carbamoyl, (vi) carboxy, (vii) C₁₋₄ alkoxy-carbonyl, (viii) sulfo, (ix) halogen, (x) C₁₋₄ alkoxy, (xi) phenoxy, (xii) halophenoxy, (xiii) C₁₋₄ alkylthio, (xiv) mercapto, (xv) phenylthio, (xvi) pyridylthio, (xvii) C₁₋₄ alkylsulfonyl, (xviii) C₁₋₄ alkylsulfonyl, (xix) amino, (xx) C₁₋₃ alkanoylamino, (xxi) mono- or di-C₁₋₄ alkylamino, (xxii) 4- to 6-membered cyclic amino, (xxiii) C₁₋₃ alkanoyl, (xxiv) benzoyl and (xxv) 5- to 10-membered heterocyclic groups.

It must be noted that, as used in this specification and the appended claims, the singular forms “a,” “an,” and “the” include plural referents unless the context clearly dictates otherwise.

In describing and claiming the present invention, the following terminology will be used in accordance with the definitions set out below.

The term “alkyl”, unless otherwise stated, means a straight or branched hydrocarbon chain having 1 to 15, preferably 1 to 6 carbon atoms, and is more preferably a methyl or ethyl group.

The term “aryl”, unless otherwise stated, is used throughout the specification to mean an aromatic cyclic hydrocarbon group. An aryl having 6 to 14 carbon atoms is preferable. It may be partially saturated. Preferred examples of such aryls are phenyl and naphthyl groups.

The term “hetaryl”, unless otherwise stated, is used throughout the specification to mean a 5- or 6-membered monocyclic or heterocyclic group containing 1 to 4 hetero-atoms selected from oxygen, sulfur and nitrogen, or a fused bicyclic heterocyclic group containing 1 to 6 hetero-atoms selected from oxygen, sulfur and nitrogen, each of which may be substituted by 1 to 4 substituents selected from the group consisting of (i) halogen, (ii) C₁₋₄ alkyl, (iii) C₁₋₄ haloalkyl, (iv) C₁₋₄ haloalkoxy, (v) C₁₋₄ alkoxy, (vi) C₁₋₄ alkylthio, (vii) hydroxy, (viii) carboxy, (ix) cyano, (x) nitro, (xi) amino, (xii) mono- or di-C₁₋₄ alkylamino, (xiii) formyl, (xiv) mercapto, (xv) C₁₋₄ alkyl-carbonyl, (xvi) C₁₋₄ alkoxy-carbonyl, (xvii) sulfo, (xviii) C₁₋₄ alkylsulfonyl, (xix) carbamoyl, (xx) mono- or di-C₁₋₄ alkyl-carbamoyl, (xxi) oxo and (xxii) thioxo groups.

The term “substituted aryl” is used throughout the specification to mean: a C₆₋₁₄ aryl group which may be substituted by 1 to 4 substituents selected from the group consisting of (i) halogen, (ii) C₁₋₄ alkyl, (iii) C₁₋₄ haloalkyl, (iv) C₁₋₄ haloalkoxy, (v) C₁₋₄ alkoxy, (vi) C₁₋₄ alkylthio, (vii) hydroxy, (viii) carboxy, (ix) cyano, (x) nitro, (xi) amino, (xii) mono- or di-C₁₋₄ alkylamino, (xiii) formyl, (xiv) mercapto, (xv) C₁₋₄ alkyl-carbonyl, (xvi) C₁₋₄ alkoxy-carbonyl, (xvii) sulfo, (xviii) C₁₋₄ alkylsulfonyl, (xix) carbamoyl, (xx) mono- or di-C₁₋₄ alkyl-carbamoyl, (xxi) oxo and (xxii) thioxo groups. The aryl can be substituted at any position thereon. Accordingly when the aryl is a phenyl, the phenyl ring can be substituted at the para, meta, ortho position, and any combination thereof.

The term “substituted hetaryl” is used throughout the specification to mean hetaryl as described above may be substituted by 1 to 4 substituents selected from the group consisting of (i) halogen, (ii) C₁₋₄ alkyl, (iii) C₁₋₄ haloalkyl, (iv) C₁₋₄ haloalkoxy, (v) C₁₋₄ alkoxy, (vi) C₁₋₄ alkylthio, (vii) hydroxy, (viii) carboxy, (ix) cyano, (x) nitro, (xi) amino, (xii) mono- or di-C₁₋₄ alkylamino, (xiii) formyl, (xiv) mercapto, (xv) C₁₋₄ alkyl-carbonyl, (xvi) C₁₋₄ alkoxy-carbonyl, (xvii) sulfo, (xviii) C₁₋₄ alkylsulfonyl, (xix) carbamoyl, (xx) mono- or di-C₁₋₄ alkyl-carbamoyl, (xxi) oxo and (xxii) thioxo groups.

The term “halo” or “halogen” is used to describe a substituent being a chlorine and fluorine. Additionally, the halogen can be a bromine when functionally possible.

The compounds of the present invention may be geometric isomers or tautomers depending upon the type of substituents. The present invention also covers these isomers in separated forms and the mixtures thereof Furthermore, some of the compounds may contain an asymmetric carbon in the molecule; in such case isomers could be present. The present invention also embraces mixtures of these optical isomers and the isolated forms of the isomers.

Some of the compounds of the invention may form salts. There is no particular limitation so long as the salt forms are pharmacologically acceptable. Specific examples of acid addition salts are the salts of inorganic acids such as hydrochloric acid, hydrobromic acid, hydriodic acid, sulfuric acid, nitric acid, phosphoric acid, etc., organic acids such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, aspartic acid, glutamic acid, etc. Specific examples of basic salts include salts with inorganic bases containing metals such as sodium, potassium, magnesium, calcium, aluminum, etc., or salts with organic bases such as methylamine, ethylamine, ethanolamine, lysine, ornithine, etc. The present invention further embraces various hydrates and solvates of the compounds or salts thereof of the invention as well as polymorphisms thereof.

Hereinafter, representative processes for producing the compounds of the present invention are described. In these processes, functional groups present in the starting materials or intermediates may be suitably protected with protective groups, depending upon the kind of functional group. In view of the preparation techniques, it may be advantageous to protect the functional groups with groups that can readily be reverted to the original functional group. When required, the protective groups are removed to give the desired products. Examples of such functional groups are amino, hydroxy, carboxy groups, etc. Examples of the groups which may be used to protect these functional groups are shown in, e.g., Greene and Wuts, “Protective Groups in Organic Synthesis”, second edition.

The general procedures for synthesizing pyrazolo[3,4-b]quinolin-5-one and pyrazolo[3,4-b]pyridin-6-one compounds is illustrated as follows:

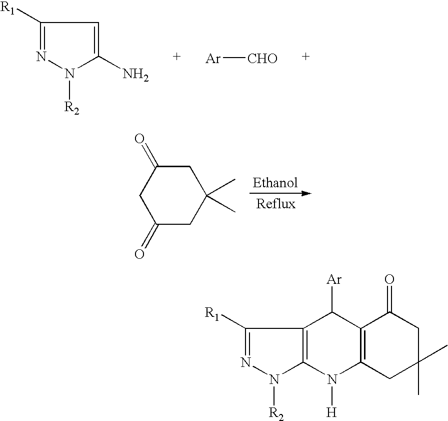

The reaction vessel was charged with aminopyrazole (1.0 mmol) dissolved in ethyl alcohol (10 mL). The appropriate aldehyde (1.0 mmol) and dimedone (1.0 mmol) were added to the above solution while stirring at room temperature. The reaction mixture was heated to 80° C. and refluxed for 6-8 h. The reaction vessel was then cooled to room temperature, and the solvent was removed under reduced pressure on a rotary evaporator. The residue was triturated with n-hexane in order to induce crystallization. The solid product was filtered off, washed abundantly with n-hexane and dried under ambient conditions. Yield: 30-75% Purity: 90-95%.

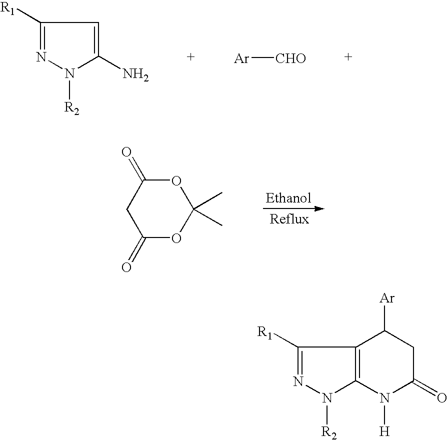

The reaction vessel was charged with aminopyrazole (1.0 mmol) dissolved in ethyl alcohol (10 mL). The appropriate aldehyde (1.0 mmol) and Meldrum's acid (1.0 mmol) were added to the above solution while stirring at room temperature. The reaction mixture was heated to 80° C. and refluxed for 6-8 h. The reaction vessel was then cooled to room temperature, and the solvent was removed under reduced pressure on a rotary evaporator. The residue was purified by flash column chromatography. Yield: 50-75% Purity: 90-95%.

The desired compound of the present invention may also be prepared by functional group transformation methods well known to those skilled in the art, which may depend on the kind of substituent. The order of the reactions, or the like, may be appropriately changed in accordance with the aimed compound and the type of reaction to be employed. The other compounds of the present invention and starting compounds can be easily produced from suitable materials in the same manner as in the above processes or by methods well known to those skilled in the art. Each of the reaction products obtained by the aforementioned production methods are isolated and purified as the free base or salt thereof. The salt can be produced by usual salt forming methods. The isolation and purification steps are carried out by employing conventional chemical techniques such as extraction, concentration, evaporation, crystallization, filtration, recrystallization, various types of chromatography and the like.

Various forms of isomers can be isolated by conventional procedures making use of physicochemical differences among isomers. For instance, racemic compounds can be separated by means of conventional optical resolution methods (e.g., by forming diastereomer salts with a conventional optically active acid such as tartaric acid, etc. and then optically resolving the salts) to give optically pure isomers. A mixture of diastereomers can be separated by conventional means, e.g., fractional crystallization or chromatography. In addition, an optical isomer can also be synthesized from an appropriate optically active starting compound.

Table 1 lists the structure of representative compounds
of the present invention.

| ID | Structure |
|---|---|
| 900658 | 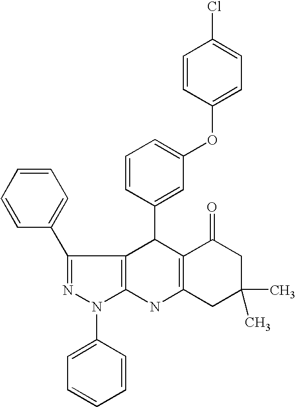 |
| 900661 | 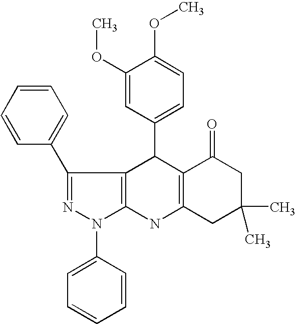 |
| 900664 | 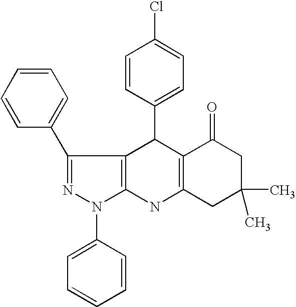 |
| 963814 | 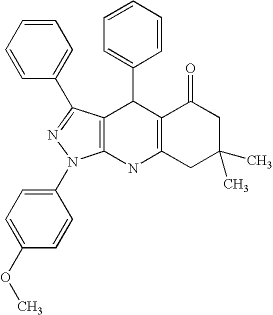 |
| 963820 | 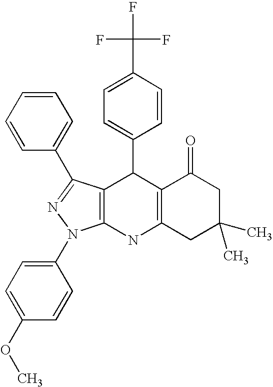 |
| 963822 | 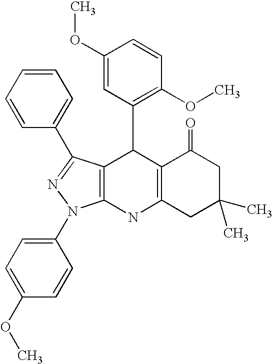 |
| 963870 | 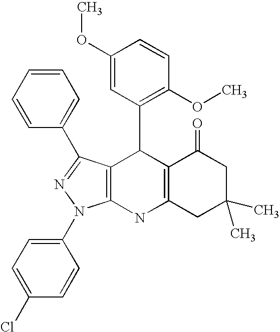 |
| 963871 | 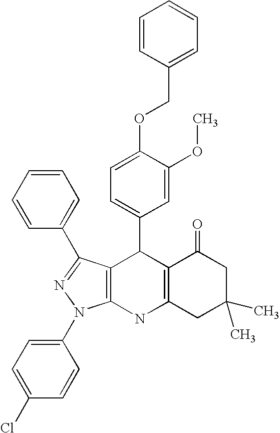 |
| 963876 | 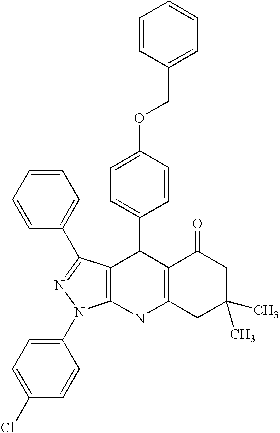 |
| 963923 | 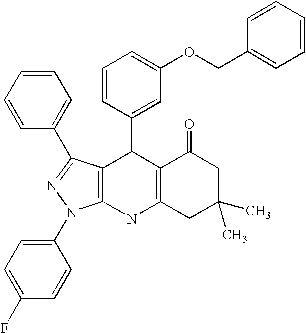 |
| 963924 | 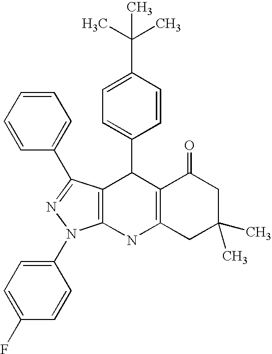 |
| 963948 | 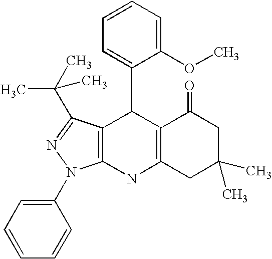 |
| 963961 | 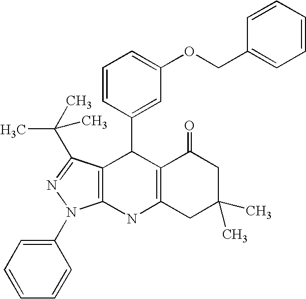 |
| 963971 | 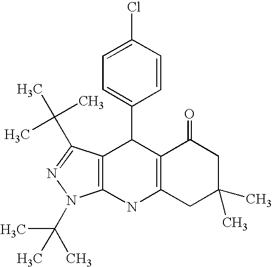 |
| 963972 | 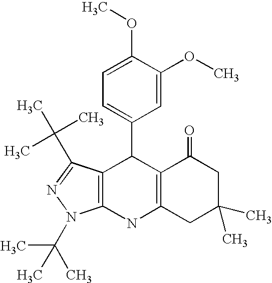 |
| 963977 | 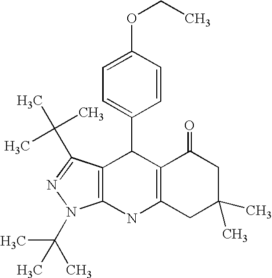 |
| 963978 | 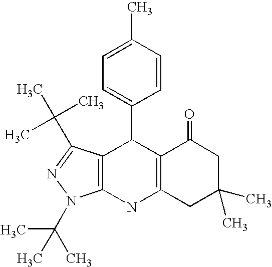 |
| 963985 | 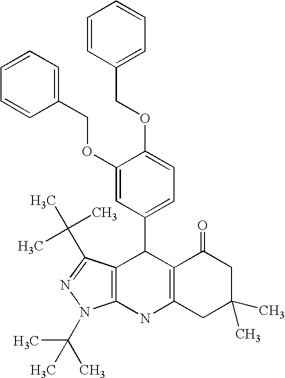 |
| 964026 | 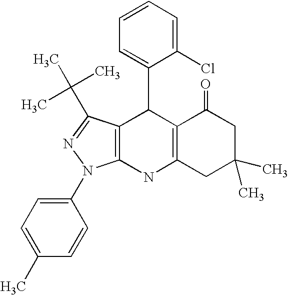 |
| 964028 | 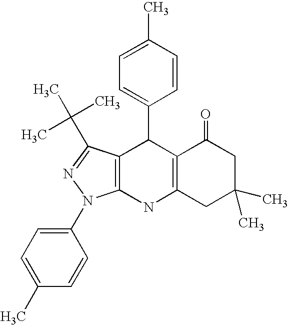 |
| 964049 | 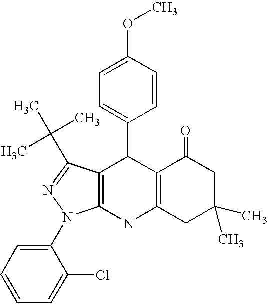 |
| 964053 | 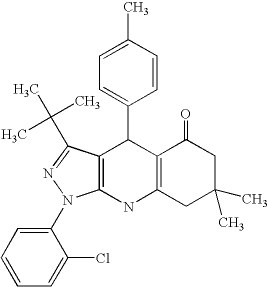 |
| 964066 | 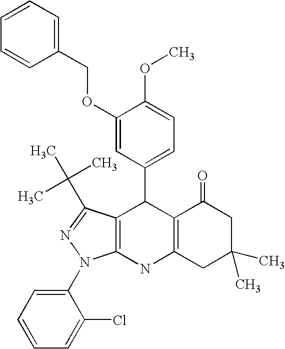 |
| 964076 | 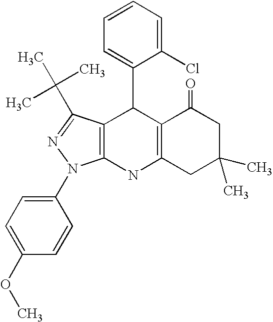 |
| 964081 | 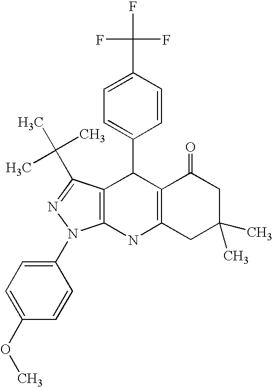 |
| 964085 | 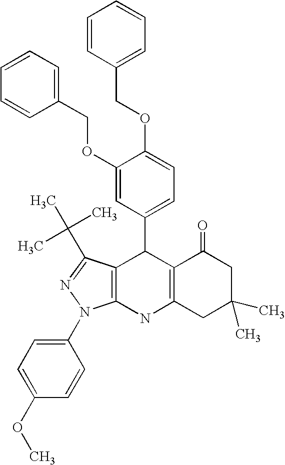 |
| 964122 | 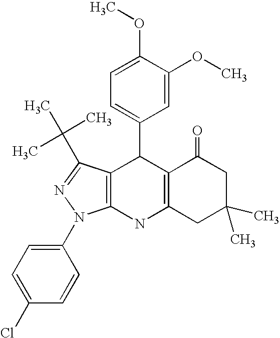 |
| 964127 | 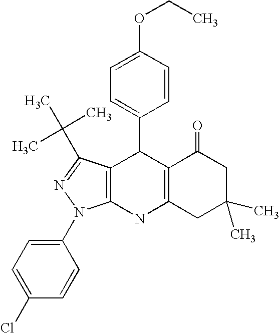 |
| 964144 | 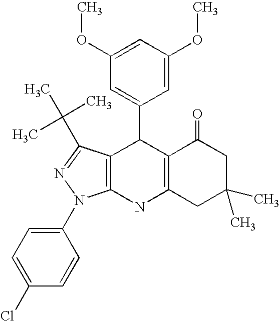 |
| 964165 | 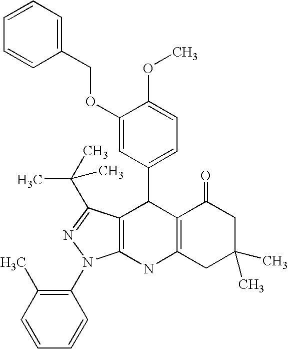 |
| 964178 | 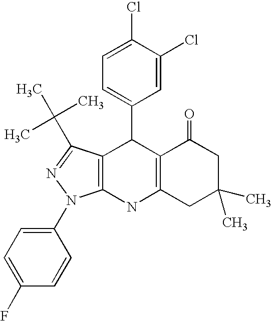 |
| 964180 | 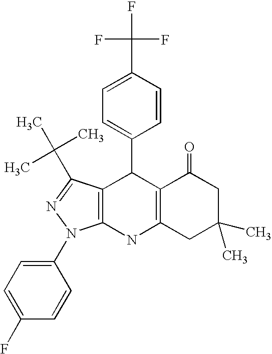 |
| 964182 | 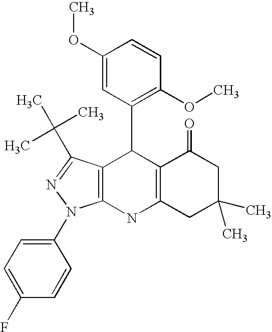 |
| 964184 | 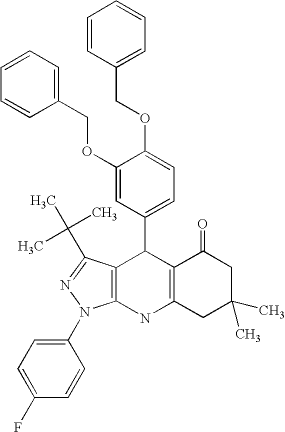 |
| 964232 | 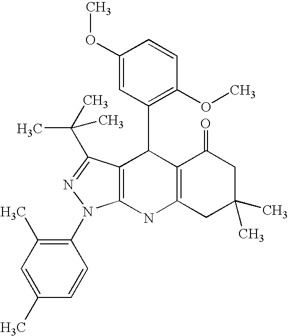 |
| 964238 | 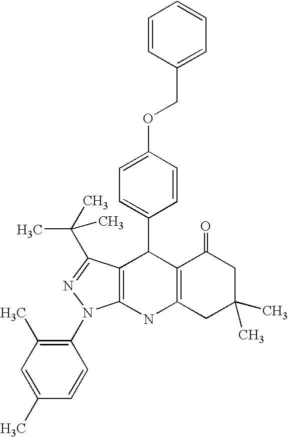 |
| 964239 | 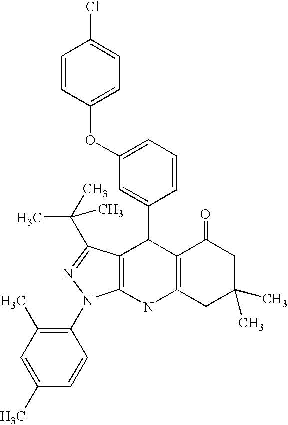 |
| 964247 | 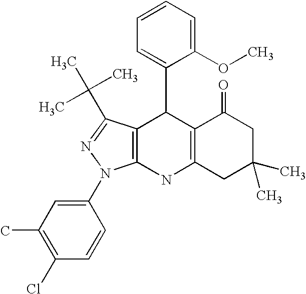 |
| 964250 | 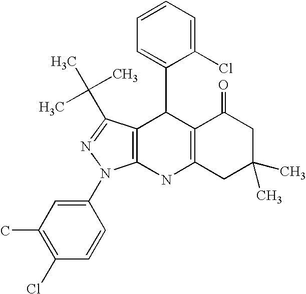 |
| 964254 | 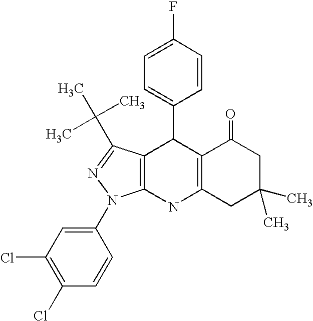 |
| 964260 | 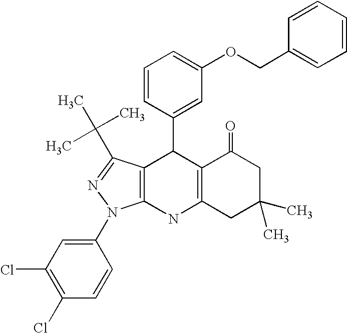 |
| 964323 | 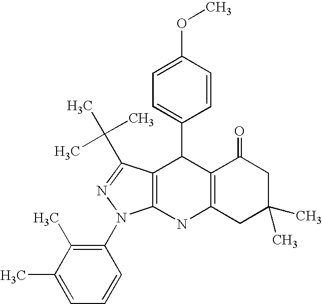 |
| 964325 | 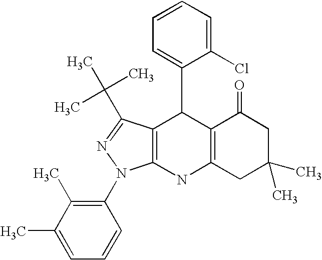 |
| 964330 | 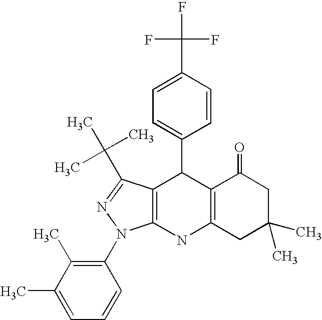 |
| 964336 | 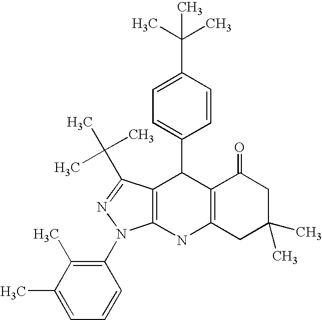 |
| 964346 | 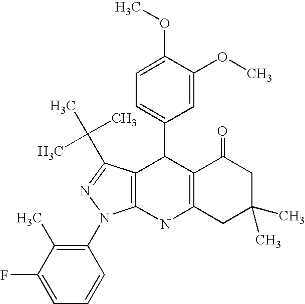 |
| 964351 | 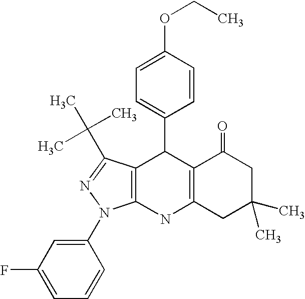 |
| 964352 | 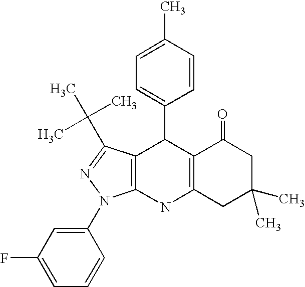 |
| 964370 | 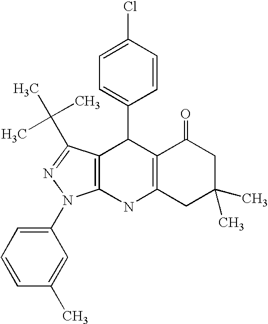 |
| 964371 | 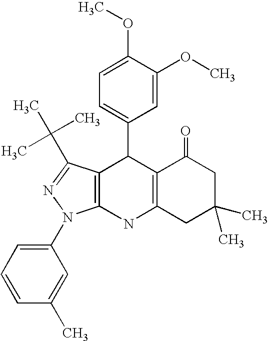 |
| 964376 | 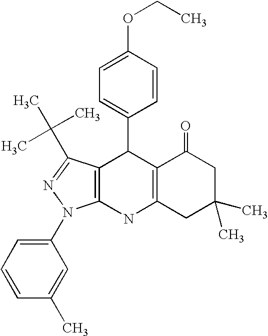 |
| 964430 | 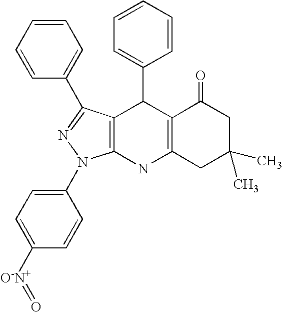 |
| 964452 | 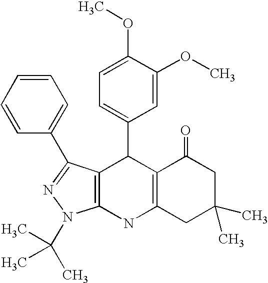 |
| 964460 | 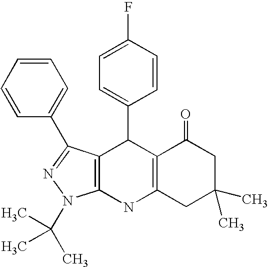 |
| 964469 | 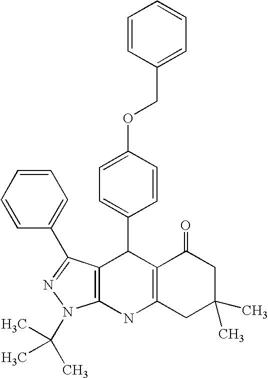 |
| 964474 | 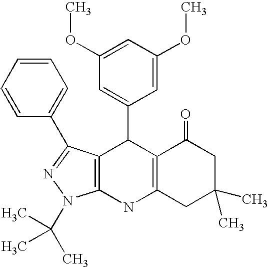 |
| 964528 | 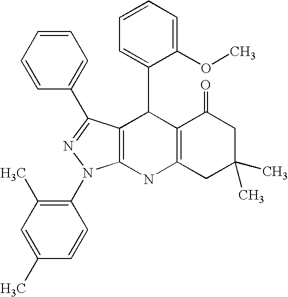 |
| 964534 | 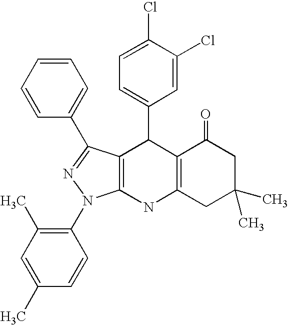 |
| 964535 | 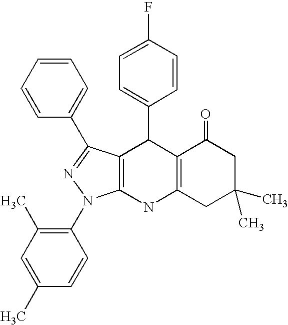 |
| 964536 | 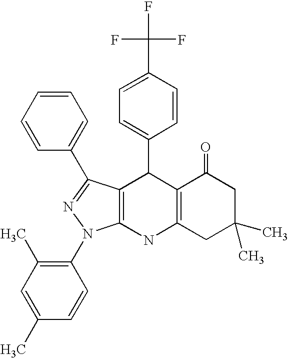 |
| 964540 | 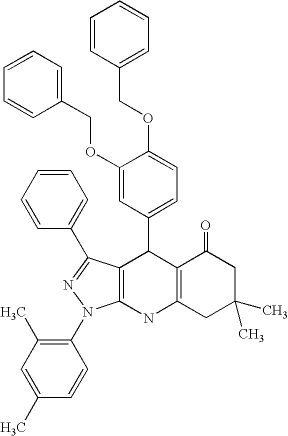 |
| 964544 | 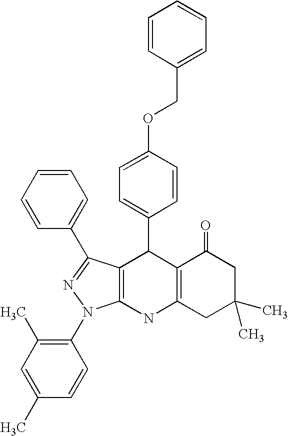 |
| 964630 | 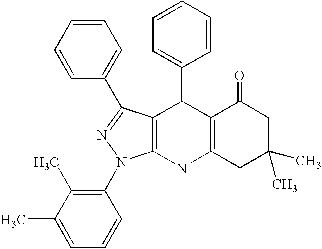 |
| 964632 | 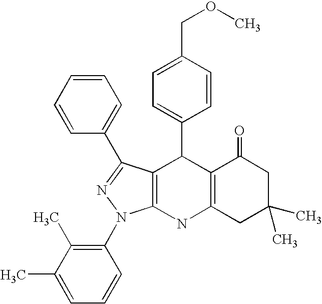 |
| 964638 | 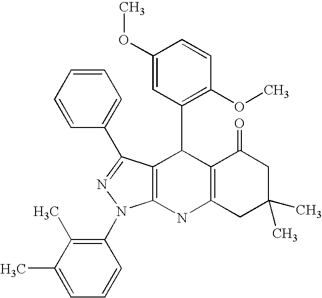 |
| 964652 | 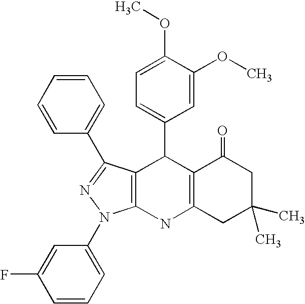 |
| 964656 | 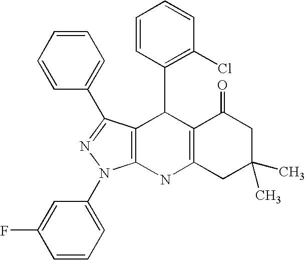 |
| 964657 | 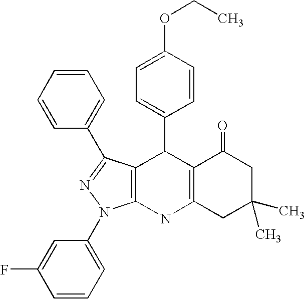 |
| 964661 | 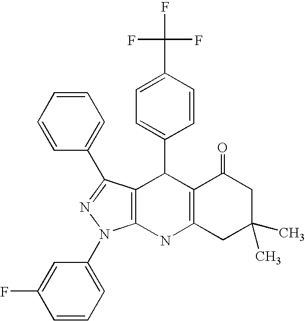 |
| 964663 | 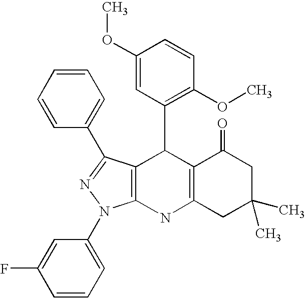 |
| 964665 | 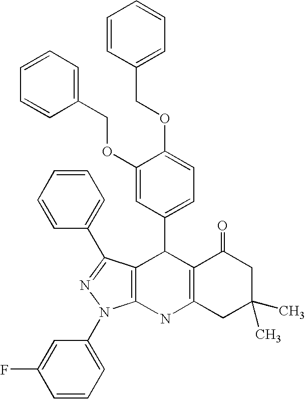 |
| 964669 | 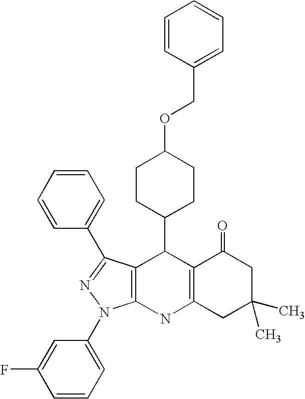 |
| 964709 | 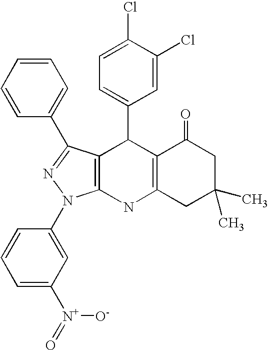 |
| 964711 | 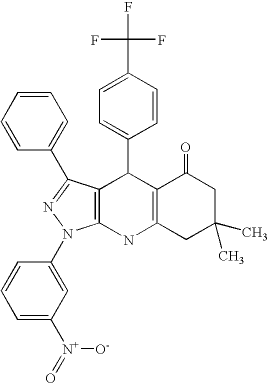 |
| 964713 | 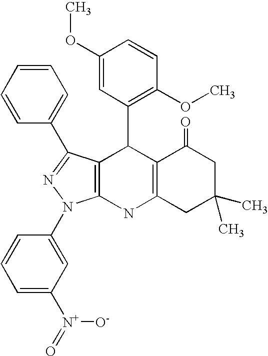 |
| 964721 | 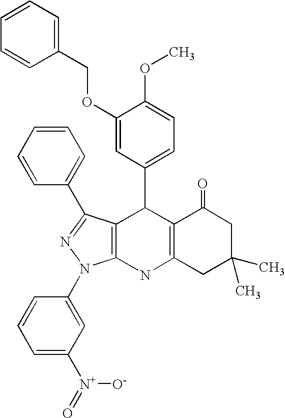 |
| 964451 | 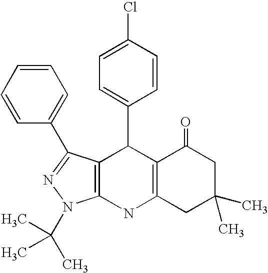 |
| 964088 | 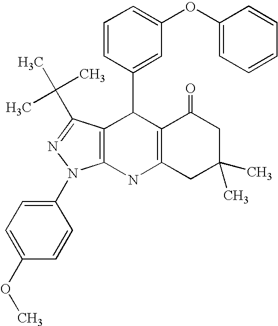 |

One embodiment of the present invention relates to compounds that inhibit the activity of PI 3-K alpha. The invention further provides methods of inhibiting PI 3-K alpha activity, including methods of modulating the activity of the PI 3-K alpha in cells, especially cancer cells. Of particular benefit are methods of modulating PI 3-K alpha activity in the clinical setting in order to ameliorate disease or disorders mediated by PI 3-K alpha activity. Thus, treatment of diseases or disorders characterized by excessive or inappropriate PI 3-K alpha activity can be treated through use of modulators of PI 3-K alpha according to the present invention.

The compounds of the present invention may also show inhibitory activity against other PI 3-K isoforms, including PI 3-K beta, gamma, and delta. Therefore, the present invention also provides methods enabling the further characterization of the physiological role of each PI 3-K isozyme. Moreover, the invention provides pharmaceutical compositions comprising PI 3-K inhibitors and methods of manufacturing and using such PI 3-Kinhibitor compounds.

The methods described herein benefit from the use of compounds that inhibit, and preferably specifically inhibit, the activity of a PI 3-K isoform in cells. Cells useful in the methods include those that express endogenous PI 3-K, wherein endogenous indicates that the cells express PI 3-K absent recombinant introduction into the cells of one or more polynucleotides encoding a PI 3-K isoform polypeptide or a biologically active fragment thereof. Methods also encompass use of cells that express exogenous PI 3-K isoforms wherein one or more polynucleotides encoding a PI 3-K isoforms or a biologically active fragment thereof, have been introduced into the cell using recombinant procedures.

Of particular advantage, the cells can be in vivo, i.e., in a living subject, e.g., an animal or human, wherein a PI 3-K inhibitor can be used therapeutically to inhibit PI 3-K activity in the subject. Alternatively, the cells can be isolated as discrete cells or in a tissue, for ex vivo or in vitro methods. In vitro methods also encompassed by the invention can comprise the step of contacting a PI 3-K enzyme, or a biologically active fragment thereof, with an inhibitor compound of the invention. The PI 3-K enzyme can include a purified and isolated enzyme, wherein the enzyme is isolated from a natural source (e.g., cells or tissues that normally express a PI 3-K polypeptide absent modification by recombinant technology) or isolated from cells modified by recombinant techniques to express exogenous enzyme.

The relative efficacies of compounds as inhibitors of enzymes activity (or other biological activity) can be established by determining the concentrations at which each compound inhibits the activity to a predefined extent and then comparing the results.

Typically, the preferred determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity are then plotted against the inhibitor concentrations used. The concentration of the inhibitor that allows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

The compounds of the present invention exhibit kinase inhibitory activity, especially PI 3-K inhibitory activity and therefore, can be utilized to inhibit abnormal cell growth in which PI 3-K plays a role. Thus, the compounds are effective in the treatment of disorders with which abnormal cell growth actions of PI 3-K are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterine cancer, ovarian cancer, prostate cancer, lung cancer, colon cancer, pancreatic cancer, renal cancer, gastric cancer, brain tumors, etc.

Accordingly, the invention provides methods of characterizing the potency of a test compound as an inhibitor of the PI 3-K polypeptide, said method comprising the steps of (a) measuring the activity of a PI 3-K polypeptide in the presence of a test compound; (b) comparing the activity of the PI3 polypeptide in the presence of the test compound to the activity of the PI 3-K polypeptide in the presence of an equivalent amount of a reference compound (e.g., a PI 3-Kα inhibitor compound of the invention as described herein), wherein lower activity of the PI 3-K polypeptide in the presence of the test compound than in the presence of the reference compound indicates that the test compound is a more potent inhibitor than the reference compound, and higher activity of the PI 3-K polypeptide in the presence of the test compound than in the presence of the reference compound indicates that the test compound is a less potent inhibitor than the reference compound.

The invention further provides methods of characterizing the potency of a test compound as an inhibitor of the PI 3-K polypeptide, comprising the steps of (a) determining the amount of a control compound (e.g., a PI 3-K alpha inhibitor compound of the invention as described herein) that inhibits an activity of a PI 3-K polypeptide by a reference percentage of inhibition, thereby defining a reference inhibitory amount for the control compound; (b) determining the amount of a test compound that inhibits the activity of a PI 3-K polypeptide by a reference percentage of inhibition, thereby defining a reference inhibitory amount for the test compound; (c) comparing the reference inhibitory amount for the test compound to the reference inhibitory amount for the control compound, wherein a lower reference inhibitory amount for the test compound than for the control compound indicates that the test compound is a more potent inhibitor than the control compound, and a higher reference inhibitory amount for the test compound than for the control compound indicates that the test compound is a less potent inhibitor than the control compound.

In one aspect, the method uses a reference inhibitory amount which is the amount of the compound than inhibits the activity of the PI 3-K alpha polypeptide by 50%, 60%, 70%, or 80%. In another aspect the method employs a reference inhibitory amount that is the amount of the compound that inhibits the activity of the PI 3-K alpha polypeptide by 90%, 95%, or 99%. These methods comprise determining the reference inhibitory amount of the compounds in an in vitro biochemical assay, in an in vitro cell-based assay, or in an in vivo assay.

The invention further provides methods of identifying a negative regulator of PI 3-K alpha activity, comprising the steps of (i) measuring activity of a PI3 alpha polypeptide in the presence and absence of a test compound, and (ii) identifying as a negative regulator a test compound that decreases PI 3-K alpha activity and that competes with a compound of the invention for binding to PI 3-K alpha. Furthermore, the invention provides methods for identifying compounds that inhibit PI 3-K alpha activity, comprising the steps of (i) contacting a PI 3-K alpha polypeptide with a compound of the invention in the presence and absence of a test compound, and (ii) identifying a test compound as a negative regulator of PI 3-K alpha activity wherein the compound competes with a compound of the invention for binding to PI 3-K alpha. The invention therefore provides a method for screening for candidate negative regulators of PI 3-K alpha activity and/or to confirm the mode of action of candidates as negative regulators. Such methods can be employed against other PI 3-K isoforms in parallel to establish comparative activity of the test compound across the isoforms and/or relative to a compound of the invention.

In these methods, the PI 3-K polypeptide can be a fragment of the peptide that exhibits kinase activity or a fragment from the binding domain that provides a method to identify allosteric modulators of the peptide. The methods can be employed in cells expressing PI 3-K peptide or its subunits, either endogenously or exogenously. Accordingly, the polypeptide employed in such methods can be free in solution, affixed to a solid support, modified to be displayed on a cell surface, or located intracellularly. The modulation of activity or the formation of binding complexes between the PI 3-K polypeptide and the agent being tested then can be measured.

Human PI 3-K polypeptides are amenable to biochemical or cell-based high throughput screening (HTS) assays according to methods known and practiced in the art, including melanophore assay systems to investigate receptor-ligand interactions, yeast-based assay systems, and mammalian cell expression systems. For a review, see Jayawickreme and Kost, Curr Opin Biotechnol, 8:629-34 (1997). Automated and miniaturized HTS assays also are comprehended as described, for example, in Houston and Banks, Curr Opin Biotechnol, 8:734-40 (1997). Such HTS assays are used to screen libraries of compounds to identify particular compounds that exhibit a desired property. Any library of compounds can be used, including chemical libraries, natural product libraries, and combinatorial libraries comprising random or designed oligopeptides, oligonucleotides, or other organic compounds.

The present invention also provides a method for inhibiting PI 3-K activity therapeutically or prophylactically. The method comprises administering an inhibitor of PI 3-K activity in an amount effective therefor in treating humans or animals who are or can be subject to any condition whose symptoms or pathology is mediated by PI 3-Kexpression or activity.

“Treating” as used herein refers to preventing a disorder from occurring in an animal that can be predisposed to the disorder, but has not yet been diagnosed as having it; inhibiting the disorder, i.e., arresting its development; relieving the disorder, i.e., causing its regression; or ameliorating the disorder, i.e., reducing the severity of symptoms associated with the disorder. “Disorder” is intended to encompass medical disorders, diseases, conditions, syndromes, and the like, without limitation.

The methods of the invention embrace various modes of treating an animal subject, preferably a mammal, more preferably a primate, and still more preferably a human. Among the mammalian animals that can be treated are, for example, companion animals (pets), including dogs and cats; farm animals, including cattle, horses, sheep, pigs, and goats; laboratory animals, including rats, mice, rabbits, guinea pigs, and nonhuman primates, and zoo specimens. Nonmammalian animals include, for example, birds, fish, reptiles, and amphibians.

In one aspect, the method of the invention can be employed to treat subjects therapeutically or prophylactically who have or can be subject to an inflammatory disorder. One aspect of the present invention derives from the involvement of PI 3-K in mediating aspects of the inflammatory process. Without intending to be bound by any theory, it is theorized that, because inflammation involves processes are typically mediated by leukocyte (e.g., neutrophils, lymphocyte, etc.) activation and chemotactic transmigration, and because PI 3-K can mediate such phenomena, antagonists of PI 3-K can be used to suppress injury associated with inflammation.

“Inflammation” as used herein refers to a localized, protective response elicited by injury or destruction of tissues, which serves to destroy, dilute, or wall off (sequester) both the injurious agent and the injured tissue. Inflammation is notably associated with influx of leukocytes and/or neutrophil chemotaxis. Inflammation can result from infection with pathogenic organisms and viruses and from noninfectious means such as trauma or reperfusion following myocardial infarction or stroke, immune response to foreign antigen, and autoimmune responses. Accordingly, inflammatory disorders amenable to the invention encompass disorders associated with reactions of the specific defense system as well as with reactions of the nonspecific defense system.

The therapeutic methods of the present invention include methods for the treatment of disorders associated with inflammatory cell activation. “Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes (i.e., polymorphonuclear leukocytes such as neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory disorder.

In a further aspect, the invention includes methods of using PI 3-K inhibitory compounds to inhibit the growth or proliferation of cancer cells of hematopoietic origin, preferably cancer cells of lymphoid origin, and more preferably cancer cells related to or derived from B lymphocytes or B lymphocyte progenitors. Cancers amenable to treatment using the methods of the present invention include, without limitation, lymphomas, e.g., malignant neoplasms of lymphoid and reticuloendothelial tissues, such as Burkitt's lymphoma, Hodgkins' lymphoma, non-Hodgkins lymphomas, lymphocytic lymphomas and the like; multiple myelomas; as well as leukemias such as lymphocytic leukemias, chronic myeloid (myelogenous) leukemias, and the like.

A compound of the present invention can be administered as the neat chemical, but it is typically preferable to administer the compound in the form of a pharmaceutical composition or formulation. Accordingly, the present invention also provides pharmaceutical compositions that comprise a chemical or biological compound (“agent”) that is active as a modulator of PI 3-K activity and a biocompatible pharmaceutical carrier, adjuvant, or vehicle. The composition can include the agent as the only active moiety or in combination with other agents, such as oligo- or polynucleotides, oligo- or polypeptides, drugs, or hormones mixed with excipient(s) or other pharmaceutically acceptable carriers. Carriers and other ingredients can be deemed pharmaceutically acceptable insofar as they are compatible with other ingredients of the formulation and not deleterious to the recipient thereof.

Techniques for formulation and administration of pharmaceutical compositions can be found in Remington's Pharmaceutical Sciences, 18th Ed., Mack Publishing Co, Easton, Pa., 1990. The pharmaceutical compositions of the present invention can be manufactured using any conventional method, e.g., mixing, dissolving, granulating, dragee-making, levigating, emulsifying, encapsulating, entrapping, melt-spinning, spray-drying, or lyophilizing processes. However, the optimal pharmaceutical formulation will be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions are formulated to contain suitable pharmaceutically acceptable carriers, and can optionally comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solution, or physiologically buffered saline. For tissue or cellular administration, penetrants appropriate to the particular barrier to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT.RTM. series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the active agent also can be employed for parenteral administration. Liposomes generally are derived from phospholipids or other lipid substances. The compositions in liposome form can also contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

The pharmaceutical compositions comprising the agent in dosages suitable for oral administration can be formulated using pharmaceutically acceptable carriers well known in the art. The preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after addition of suitable auxiliaries if desired, to obtain tablets or dragee cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or more excipients, which include, without limitation:
a) diluents, such as sugars, including lactose, dextrose, sucrose, mannitol, or sorbitol;
b) binders, such as magnesium aluminum silicate, starch from corn, wheat, rice, potato, etc.;
c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;
d) disintegrating or solubilizing agents such as cross-linked polyvinyl pyrrolidone, starches, agar, alginic acid or a salt thereof, such as sodium alginate, or effervescent compositions;
e) lubricants, such as silica, talc, stearic acid or its magnesium or calcium salt, and polyethylene glycol;
f) flavorants and sweeteners;
g) colorants or pigments, e.g., to identify the product or to characterize the quantity (dosage) of active compound; and
h) other ingredients, such as preservatives, stabilizers, swelling agents, emulsifying agents, solution promoters, salts for regulating osmotic pressure, and buffers.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragee cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a salt of the active agent. Salts tend to be more soluble in aqueous or other protonic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metals (e.g., sodium or potassium) and alkaline earth (e.g., calcium or magnesium) cations.

Compounds of structural formula I-III of the present invention can form pharmaceutically acceptable acid addition salts with suitable acids. For example, Berge et al,. describe pharmaceutically acceptable salts in detail in J Pharm Sci, 66:1 (1977). The salts can be prepared in situ during the final isolation and purification of the compounds of the invention or separately by reacting a free base function with a suitable acid.

In light of the foregoing, any reference to compounds of the present invention appearing herein is intended to include compounds of structural formula described above as well as pharmaceutically acceptable salts and solvates, as well as prodrugs thereof.

Compositions comprising a compound of the present invention formulated in a pharmaceutically acceptable carrier can be prepared, placed in an appropriate container, and labeled for treatment of an indicated condition. Accordingly, there also is contemplated an article of manufacture, such as a container comprising a dosage form of a compound of the invention and a label containing instructions for use of the compound. Kits are also contemplated under the invention. For example, the kit can comprise a dosage form of a pharmaceutical composition and a package insert containing instructions for use of the composition in treatment of a medical condition. In either case, conditions indicated on the label can include treatment of inflammatory disorders, cancer, etc.

Pharmaceutical compositions comprising an inhibitor of PI 3-K activity can be administered to the subject by any conventional method, including by parenteral and enteral techniques. Parenteral administration modalities include those in which the composition is administered by a route other than through the gastrointestinal tract, for example, by intravenous, intraarterial, intraperitoneal, intramedullary, intramuscular, intraarticular, intrathecal, and intraventricular injections. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments. Parenteral administration also can be accomplished using high-pressure techniques.

Surgical techniques include implantation of depot (reservoir) compositions, osmotic pumps, and the like. A preferred route of administration for treatment of inflammation can be local or topical delivery for localized disorders such as arthritis, or systemic delivery for distributed disorders, e.g., intravenous delivery for reperfusion injury or for systemic conditions such as septicemia. For other diseases, including those involving the respiratory tract, e.g., chronic obstructive pulmonary disease, asthma, and emphysema, administration can be accomplished by inhalation or deep lung administration of sprays, aerosols, powders, and the like.

For the treatment of neoplastic diseases, especially leukemias and other distributed cancers, parenteral administration is typically preferred. Formulations of the compounds to optimize them for biodistribution following parenteral administration would be desirable. The PI 3-K inhibitor compounds can be administered before, during, or after administration of chemotherapy, radiotherapy, and/or surgery.

As noted above, the characteristics of the agent itself and the formulation of the agent can influence the physical state, stability, rate of in vivo release, and rate of in vivo clearance of the administered agent. Such pharmacokinetic and pharmacodynamic information can be collected through preclinical in vitro and in vivo studies, later confirmed in humans during the course of clinical trials. Thus, for any compound used in the method of the invention, a therapeutically effective dose can be estimated initially from biochemical and/or cell-based assays. Then, the dosage can be formulated in animal models to achieve a desirable circulating concentration range that modulates PI 3-Kexpression or activity. As human studies are conducted, further information will emerge regarding the appropriate dosage levels and duration of treatment for various diseases and conditions.

Toxicity and therapeutic efficacy of such compounds can be determined by standard pharmaceutical procedures using cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index,” which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosages for human use. The dosage of such compounds lies preferably within a range of circulating concentrations that include the ED₅₀ with little or no toxicity.

For the methods of the present invention, any effective administration regimen regulating the timing and sequence of doses can be used. Doses of the agent preferably include pharmaceutical dosage units comprising an effective amount of the agent. As used herein, “effective amount” refers to an amount sufficient to modulate PI 3-K expression or activity and/or derive a measurable change in a physiological parameter of the subject through administration of one or more of the pharmaceutical dosage units.

Exemplary dosage levels for a human subject are on the order of from about 0.001 milligram of active agent per kilogram body weight (mg/kg) to about 100 mg/kg. Typically, dosage units of the active agent comprise from about 0.01 mg to about 10,000 mg, preferably from about 0.1 mg to about 1,000 mg, depending upon the indication, route of administration, etc. Depending on the route of administration, a suitable dose can be calculated according to body weight, body surface area, or organ size. The final dosage regimen will be determined by the attending physician in view of good medical practice, considering various factors that modify the action of drugs, e.g., the agent's specific activity, the identity and severity of the disease state, the responsiveness of the patient, the age, condition, body weight, sex, and diet of the patient, and the severity of any infection.

Additional factors that can be taken into account include time and frequency of administration, drug combinations, reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials. Appropriate dosages can be ascertained through use of established assays for determining concentration of the agent in a body fluid or other sample together with dose response data.

The frequency of dosing will depend on the pharmacokinetic parameters of the agent and the route of administration. Dosage and administration are adjusted to provide sufficient levels of the active moiety or to maintain the desired effect. Accordingly, the pharmaceutical compositions can be administered in a single dose, multiple discrete doses, by continuous infusion, as sustained release depots, or combinations thereof, as required to maintain the desired minimum level of the agent. Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks. Pumps, such as subcutaneous, intraperitoneal, or subdural pumps, can be preferred for continuous infusion.

The following Examples are provided to further aid in understanding the invention, and pre-suppose an understanding of conventional methods well-known to those persons having ordinary skill in the art to which the examples pertain. Such methods are described in detail in numerous publications including, for example, Sambrook et al., Molecular Cloning: A Laboratory Manual, Cold Spring Harbor Laboratory Press (1989), Ausubel et al. (Eds.), Current Protocols in Molecular Biology, John Wiley & Sons, Inc. (1994); and Ausubel et al. (Eds.), Short Protocols in Molecular Biology, 4th ed., John Wiley & Sons, Inc. (1999). The particular materials and conditions described hereunder are intended to exemplify particular aspects of the invention and should not be construed to limit the reasonable scope thereof.

### EXAMPLE 1

### Synthesis and Characterization of 964076 3-t-butyl-4-(2-chlorophenyl)-7,7-dimethyl-1-(4-methoxyphenyl)-4,7,8,9-tetrahydro-1H-pyrazolo[3,4-b]quinolin-5(6H)-one

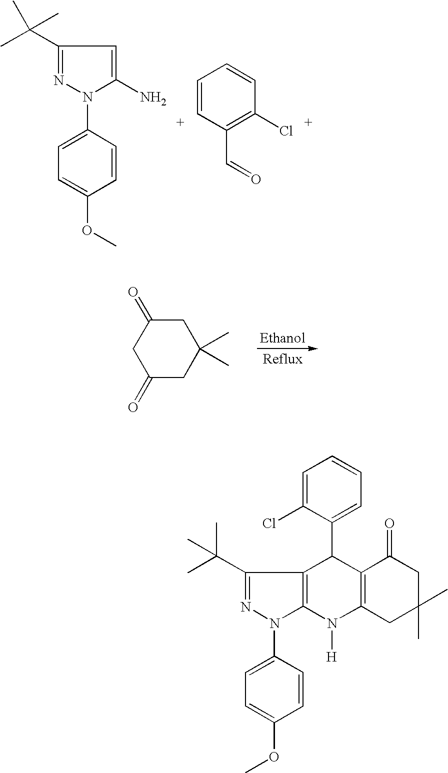

The reaction vessel was charged with 1-(4-methoxyphenyl)-3-t-butyl-5-aminopyrazole (500 mg, 2.03 mmol) dissolved in ethyl alcohol (20 mL). Then, (2-chloro-benzaldehyde (218 mL, 2.43 mmol) and dimedone (285 mg, 1.0 mmol) were added to the above solution while stirring at room temperature. The reaction mixture was heated to 80° C. and refluxed for 6 h.

The reaction vessel was then cooled to room temperature, and the solvent was removed under reduced pressure on a rotary evaporator. The residue was triturated with n-hexane in order to induce crystallization. The solid product was re-dissolved and further purified by column chromatography yielding a pure product (110 mg) which was then characterized by NMR:

1H NMR (CDCl3): 0.8, 1.02, 1.1, 1.23, 2.03, 2.14, 3.85, 5.67, 6.32, 7.02, 7.14, 7.23, 7.44.

### EXAMPLE 2

### Synthesis and Characterization of 964028 3-t-butyl-4-(4-methylphenyl)-7,7-dimethyl-1-(4-methylphenyl)-4,7,8,9-tetrahydro-1H-pyrazolo[3,4-b]quinolin-5(6H)-one

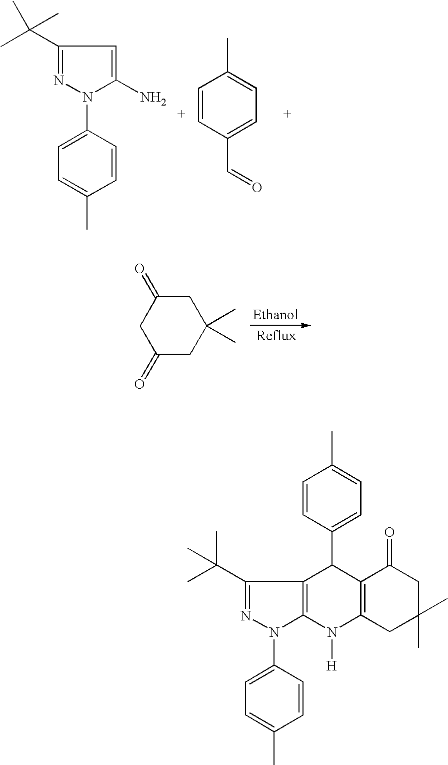

The reaction vessel was charged with l-(4-methylphenyl)-3-t-butyl-5-aminopyrazole (180 mg, 0.78 mmol) dissolved in ethyl alcohol (10 mL). A p-tolualdehyde (110 mg, 0.94 mmol) and dimedone (110 mg, 0.78 mmol) were then added to the above solution while stirring at room temperature. The reaction mixture was heated to 80° C. and refluxed for 6 h. The reaction vessel was then cooled to room temperature, and the solvent was removed under reduced pressure on a rotary evaporator. The residue was triturated with n-hexane in order to induce crystallization. The solid product (178 mg) was filtered off, washed and dried under ambient conditions which was then characterized by NMR: 1H NMR (CDCl3): 0.82, 1.03, 1.14, 1.23, 2.25, 2.41, 5.40, 6.22, 7.00, 7.18, 7.31, 7.43.

### EXAMPLE 3

### Synthesis and Characterization of 1,3-Di-t-butyl-4-p-trifluoromethylphenyl-1,4,5,7-tetrahydro-pyrazolo[3,4-b]pyridin-6-one

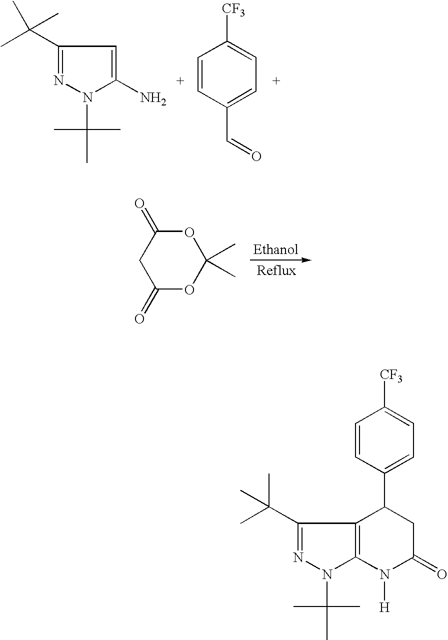

The reaction vessel was charged with 1,3-di-t-butyl-5-aminopyrazole (50 mg, 0.26 mmol) dissolved in ethyl alcohol (5 mL). p-trifluoromethylbenzaldehyde (44.6 mg, 0.26 mmol) and Meldrum's acid (36 mg, 0.26 mmol) were then added to the above solution while stirring at room temperature. The reaction mixture was heated to 80° C. and refluxed for 6 h. The reaction vessel was then cooled to room temperature, and the solvent was removed under reduced pressure on a rotary evaporator. The residue was purified by chromatography over silica, eluting with a mixture of hexanes and ethyl acetate. The solid product (70 mg) was isolated then characterized by NMR.: 1H NMR (CDCl3):1.12, 1.59, 2.67, 3.12, 4.40, 7.14, 7.51, 8.23.

### EXAMPLE 4

### Synthesis and Characterization of 1,3-Di-t-butyl-4-(3,4-dimethoxy-phenyl)-4,6,7,8-tetrahydro-1H-1,2,8-triaza-s-indacen-5-one

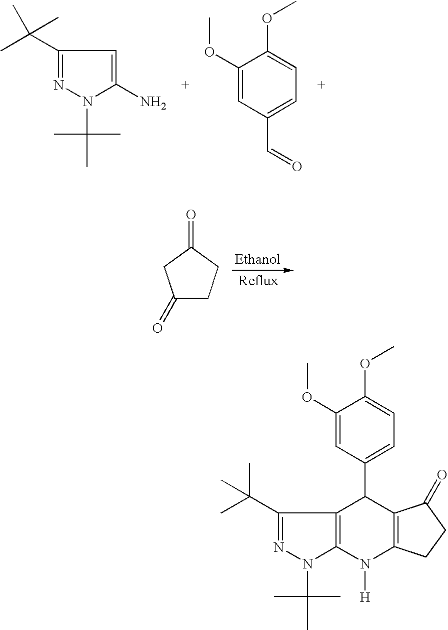

The reaction vessel was charged with 1,3-di-t-butyl-5-aminopyrazole (40 mg, 0.20 mmol) dissolved in ethyl alcohol (5 mL). 3,4-Dimethoxybenzaldehyde (34 mg, 0.20 mmol) and 1,3 cyclopentadione (36 mg, 0.26 mmol) were then added to the above solution while stirring at room temperature. The reaction mixture was heated to 80° C. and refluxed for 6 h. The reaction vessel was then cooled to room temperature, and the solvent was removed under reduced pressure on a rotary evaporator. The residue was purified by chromatography over silica, eluting with a mixture of hexanes and ethyl acetate. The solid product (60 mg) was isolated then characterized by NMR: 1H NMR (CDCl3):0.95, 1.56, 2.27, 2.49, 3.59, 3.69, 3.71, 5.00, 6.58, 3.61, 6.78.

### EXAMPLE 5

### Synthesis Characterization of 4-(3,4-Bis-benzyloxy-phenyl)-1,3-di-t-butyl-4,6,7,8,9,10-hexahydro-1H-1,2,10-triaza-cyclohepta[f]inden-5-one

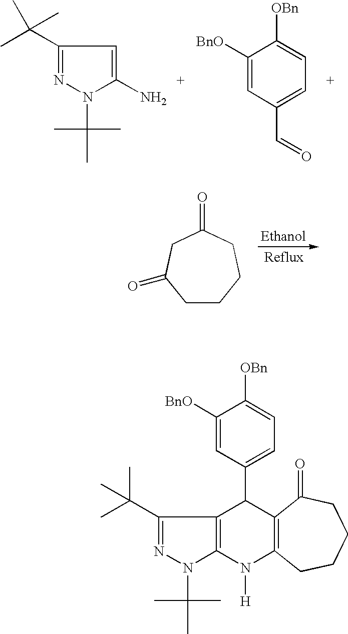

The reaction vessel was charged with 1,3-di-t-butyl-5-aminopyrazole (40 mg, 0.20 mmol) dissolved in ethyl alcohol (5 mL). 3,4-Dibenzyloxybenzaldehyde (65 mg, 0.20 mmol) and 1,3 cycloheptadione (36 mg, 0.26 mmol) were then added to the above solution while stirring at room temperature. The reaction mixture was heated to 80° C. and refluxed for 6 h. The reaction vessel was then cooled to room temperature, and the solvent was removed under reduced pressure on a rotary evaporator. The residue was purified by chromatography over silica, eluting with a mixture of hexanes and ethyl acetate. The solid product (1 8 mg) was isolated then characterized by N: 1H NMR (CDCl3):1.03, 1.64, 2.42, 5.07, 5.28, 6.72, 7.31, 7.37.

### EXAMPLE 6

### Synthesis and Characterization of 1-(1,3-Di-tert-butyl-4-p-tolyl-4,7-dihydro-1H-pyrazolo[3,4-b]pyridin-5-yl)-ethanone

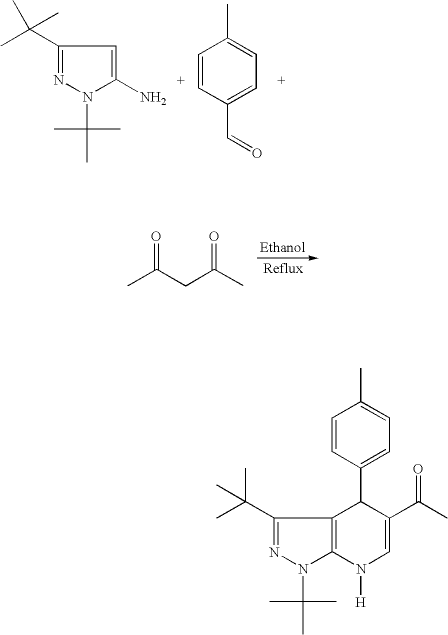

The reaction vessel is charged with 1,3-di-t-butyl-5-aminopyrazole (40 mg, 0.20 mmol) dissolved in ethyl alcohol (5 mL). p-Tolualdehyde (23 mg, 0.20 mmol) and 1,3 pentadione (36 mg, 0.26 mmol) are added to the above solution while stirring at room temperature. The reaction mixture is heated to 80° C. and refluxed for 6 hours. The reaction vessel is then cooled to room temperature, and the solvent is removed under reduced pressure on a rotary evaporator. The residue is purified by chromatography over silica, eluting with a mixture of hexanes and ethyl acetate.

### EXAMPLE 7

### Isolation and Purification of Recombinant PI 3-K Polypeptide

Recombinant heterodimeric PI 3-K alpha, consisting of a p110 catalytic subunit and a GST-tagged p85 regulatory subunit, was expressed in Sf9 cells using a baculoviras expression system. Expression constructs were obtained from the lab of Dr. Alex Toker, Harvard University. The method is well known to those skilled in the art and is also described in Stoyanov et al., Science 269, 690-693 (1995).and Stoyanova et al., Biochem. J. 324 :489-495. (1997).

The harvested cell pellet was re-suspended in 3 ml of Buffer A (20 mM Tris pH 7.0, 150 mM NaCl, 10 mM EDTA, 20 mM Sodium Fluoride, 5 mM Sodium Pyrophosphate, 10% Glycerol, 0.1% Igapal) containing protease inhibitors (1 mM PMSF, 1 mM NaVO₃, Leupeptin 1 ug/mnl, Pepstatin 1 ug/ml.) The suspension was incubated for 1 hour at 4° C. with rotation to break the cells, and then vortexed gently to ensure cell lysis. The solution was centrifuged at 14,000 g for 15 minutes, and the supernatant was diluted by the addtion of 10 ml of Buffer A. The diluted supernatant was added to 3 ml of Glutathione-agarose resin (Pharmacia) pre-equilibrated in Buffer A, and incubated for 1 hour at 4° C. with rotation. The resin was poured into a column and washed with 35 ml of Buffer A, and the protein was eluted using 10 mM Glutathione in Buffer A. Twenty, 0.5 ml fractions were collected and the presence of protein was assessed on 12% SDS-PAGE Tris Glycine gel (Invitrogen). Fractions containing target protein were pooled and concentrated using a Microsep 30K concentrator (Pall-Gelnan). The concentrated protein was diluted with 3 ml of Final Buffer (20 mM Tris pH 7.4, 100 mM NaCl, 1 mM EDTA) and concentrated twice more to remove any detergent. The protein was diluted in 50% glycerol and stored at −20° C.

### EXAMPLE 8

### PI 3-K Activity Assay and Screen for PI 3-K Inhibitors

Vectors for expression of GST-GRP1-PH were obtained from Mark Lemmon, University of Pennsylvania. (Kavran, et al., J Biol Chem, 273:30497-30508 (1998)). Protein expression and purification from E. coli was carried out as follows: A LB/amp plate was streaked from a frozen glycerol stock of E. coli containing the expression vector and grown overnight at 37° C. A single colony was picked and inoculated into 20 ml of LB media containing 100 ug/ml of ampicillin, and grown overnight. The overnight culture was added to 1 Liter of LB media containing 100 ug/ml of ampicillin and grown until the O.D. 600 was between 0.8-1.0. Protein expression was induced by the addition of 0.1 mM IPTG, and cultures continued to grow overnight at 37° C. Cells were harvested by centrifugation at 4,000 g for 20 minutes. Pellets were stored frozen at −80° C. until protein purification was carried out. The purification of GST-tagged protein was performed as follows: the pellets were resuspended in 25 ml of Buffer A (50 mM Tris pH 7.5, 1 mM BME, 1 mM EDTA, 1 mM EGTA, 1 mM NaVO3, 50 mM Sodium Fluoride, 5 mM Sodium Pyrophosphate, 0.27M Sucrose) with protease inhibitors (1 mM PMSF, 0.5 ug/ml Leupeptin, 0.7 ug/ml Pepstatin). The cells were lysed by sonication for 3 minutes, and Triton x-100 was added to a final concentration of 0.01%. The mixture was clarified by centrifugation at 10,000 rpm for 15 minutes. The supernatant was mixed with 5 ml Glutathione-agarose resin (Amersham), pre-equilibrated in Buffer A. The protein was allowed to bind to the resin for 1 hour at 4° C. with rotation. The resin was transferred into a column and washed with 30 ml of Buffer A. The protein was eluted using 10 mM Glutathione (Sigma) in Buffer A. Twenty, 1 ml fractions were collected and protein levels assessed by SDS-PAGE on 12% Tris-Glycine gels (fivitrogen). The fractions containing purified protein were pooled and stored at −20 C°.

PI 3-kinase reactions were performed in a reaction buffer containing 5 mM HEPES, pH 7, 2.5 mM MgCl₂, and 25 μM ATP, containing 50 ng of recombinant PI 3-K with 10 picomoles of diC₈ PI(4,5)P₂ (Echelon Biosciences) as the substrate. The reactions were allowed to proceed at room temperature for 1-3 hours, then quenched by the addition of EDTA to a final concentration of 10 mM. The final reaction volumes were 10 μl. The compounds to be tested for inhibition were added to a final concentration of 1 μM from stocks in DMSO. The final concentration of DMSO was 1%.

Conversion of the substrate to PI(3,4,5)P₃ was determined using a competition assay using Amplified Luminescent Proximity Homogeneous Assay (ALPHA®) technology developed by Perkin Elmer. 0.25 picomoles of recombinant GST-Grp1-PH domain protein and 0.25 picomoles of biotinylated diC₆ PI(3,4,5)P₃ (Echelon Biosciences) were added to each reaction mixture. Donor and Acceptor beads from the AlphaScreen® GST (Glutathione-S-Transferase) Detection Kit (PerkinElmer) were added to a final concentration of 20 μg/ml. The final volume was 25 μl. The reactions were incubated at 37° C. for two hours, and the luminescent signal was read on a Fusion α microplate reader. Percent inhibition of enzyme activity was determined by comparison to no enzyme (100% inhibition) and DMSO alone (0% inhibition) controls.

An alternate method used for detecting substrate conversion to PI(3,4,5)P₃ was a competitive Fluorescence Polarization assay. 125 picomoles of recombinant GST-Grp1-PH domain protein and 0.25 picomoles of TAMRA-I(1,3,4,5)P₄ (Echelon Biosciences) were added to each reaction mixture The final volume was 25 μl. Polarization values were measured on a microplate reader using 550 nm excitation/580 nm polarizing emission filters. BODIPY-TMR-I(1,3,4,5)P4 or BODIPY-TMR-PI(3,4,5)P3 could substitute as the fluorescent tracers in this assay. Percent inhibition of enzyme activity was determined by comparison to no enzyme (100% inhibition) and DMSO alone (0% inhibition) controls.

### EXAMPLE 9

### Determination of IC₅₀ for PI 3-K Inhibitors

A library of potential PI 3-K inhibitors was tested for activity against PI 3-K alpha in the following manner. From the active compounds identified, twelve were selected as representatives from different chemical groups present in the library and subjected to further analysis. IC₅₀ values were determined for the selected compounds of the present invention. Enzyme activity assays were performed as previously described, in the presence of a range of compound concentrations to allow determination of IC₅₀ values. Enzyme activity and percent inhibition was determined using the AlphaScreen® luminescent assay or a Fluorescence Polarization assay as previously described. These inhibitors may also show activity against other PI 3-K isoforms, including PI 3-K beta, gamma, and delta.

### EXAMPLE 10

### Characterization of Effects of PI 3-K Inhibitors on Cancer Cells

Selected compounds were tested for selective activity against paired ovarian cancer and breast cancer cell lines.

The ovarian cancer cell line SKOV3 is not altered in PI 3-K signaling and should be less sensitive to the anti-proliferative effects produced by treatment with PI 3-K inhibitors, while the OVCAR₃ cell line, which is altered in PI 3-K signaling, via amplification of PI 3-K activity, should be sensitive. SKOV3 cells were seeded in 96-well cell culture plates (Greiner) at a density of 20,000 cells per well in McCoys 5A media (GibcoBRL) with 10% fetal calf serum and 20 mM L-glutamine. OVCAR₃ cells were seeded at a density of 15,000 cells per well in RPMI 1640 media (GibcoBRL) containing 20 mM 1-glutamine, 0.01 ng/ml bovine insulin, 10 mM Hepes pH 7.4, 1 mM sodium pyruvate, 2.5 g/L glucose, and 20% fetal calf serum. After 24 hours, compounds were added to cell media to a final concentration of 1 μM, and the cells were grown in the presence of the compounds for 48 hours, in media containing 0.5% fetal calf serum. Viability was determined using a MTT cell proliferation assay (R and D Systems) and comparison to DMSO alone controls (100% viability). Compounds which result in reduced viability may act either by inhibiting cell proliferation or by inducing apoptosis (programmed cell death). Compounds representative of the 096 structural groups within the library showed selective effects on cell proliferation and viability.

Compounds present in the library which had been identified as PI 3-K inhibitors using the in vitro screen, and which were also structurally related to the compounds of the present invention that showed cell-specific effects on viability, were tested for activity against paired ovarian cancer cell lines. Many of these also show similar selective effects on cell growth. Table 2 summarizes the results of two separate cell proliferation experiments for selected compounds of the present invention.

Selected compounds were evaluated against paired ovarian cancer cell lines at a range of concentrations to determine effective concentrations for growth inhibition.

TABLE 2
Summary of two different experiments in which compounds of the present
invention were tested for selective effects on paired ovarian cancer cell lines.

| | Trial 1 | | Trial 2 | | Average | average |
|---|---|---|---|---|---|---|
| Compound | SKOV-3 | OVCAR3 | SKOV-3 | OVCAR3 | SKOV3 | OVCAR-3 |
| 964661 | 99 | 36 | 77.1 | 58.9 | 88.05 | 47.45 |
| 964076 | 100 | 41 | 92.9 | 53.2 | 96.45 | 47.1 |
| 964127 | 100 | 42 | 100 | 76.2 | 100 | 59.1 |
| 964144 | 100 | 54 | 100 | 66.2 | 100 | 60.1 |
| 964352 | 93 | 33 | 74 | 52.9 | 83.5 | 42.95 |
| 964028 | 100 | 42 | 99.1 | 45.4 | 99.55 | 43.7 |
| 964247 | 100 | 42 | 100 | 43.7 | 100 | 42.85 |
| 964336 | 96 | 32 | 83 | 56 | 89.5 | 44 |
| 964260 | 93 | 41 | 85 | 53 | 89 | 47 |
| 964232 | 98 | 45 | 100 | 71 | 99 | 58 |
| 963977 | 98 | 41 | 81.7 | 59.6 | 89.85 | 50.3 |
| 963924 | 100 | 61 | 100 | 66.4 | 100 | 63.7 |

PI 3-K inhibitors which show this activity profile may be effective against a number of tumor cell lines and tumor types in which PI 3-K signaling is altered, either by amplification of PI 3-K activity, or by mutations which effect regulation of PI 3-K activity, including mutations in the tumor suppressor PTEN gene. These include breast, prostate, colon, and ovarian cancers.

PI 3-K inhibitors were also evaluated for selective activity against breast cancer cell lines. The cell line MDA-MB-468 is mutant of PTEN, a negative regulator of PI 3-K signaling, and PI 3-K signaling is abnormally activated in these cells, while the cell line MDA-MB-231 shows normal expression and activity of PTEN and PI 3-K signaling is normally regulated.

MDA-MB-468 and MDA-MB-231 cells were seeded in 96-well cell culture plates (Greiner) at a density of 20,000 cells per well in RMPI media (GibcoBRL) with 10% fetal calf serum and 20 mM L-glutamine. After 24 hours, compounds were added to cell media to a final concentrations ranging from 10 nM to 100 μM, and the cells were grown in the presence of the compounds for 48 hours in RMPI media containing 0.5% fetal calf serum and 20 mM L-glutamine. Viability was determined using a MTT cell proliferation assay (R and D Systems) and comparison to DMSO alone controls (100% viability). Compounds which result in reduced viability may act either by inhibiting cell proliferation or by inducing apoptosis (programmed cell death). Compounds representative of the 096 structural groups within the library showed selective effects on cell proliferation and viability. Selected compounds were evaluated against the paired breast cancer cell lines at a range of concentrations to determine effective concentrations for growth inhibition.

### EXAMPLE 11

### Effects on PI 3-K Mediated Signaling through PKB/Akt by PI 3-K Inhibitors

Because phosphorylation and activation of PKB/Akt is dependent on PI 3-K activity, PI 3-K inhibitors decrease the cellular levels of phospho-Akt. MDA-MB-468 cells show constitutively high levels of phospho-Akt as a result of abnormal activation of PI 3-K signaling.

The effect of treatment with PI 3-K inhibitors on phospho-Akt levels in these cells was determined as follows. Cells were plated into 6-well cell culture dishes at a density of 5×10⁵ cells per well in RMPI media containing 10% fetal calf serum and 2 mM L-glutamine. Twenty-four hours later, media was removed and replaced with serum-free RMPI containing 2 mM L-glutamine. The cells were serum-starved overnight.

Compounds were diluted into serum-free media to a final concentration of 50 μM and added to the cells. The cells were incubated in the presence of PI 3-K inhibitors for 4 hours. Phospho-Akt levels were determined using one of the following methods.

To determine phospho-Akt levels using immunoblotting, cells were washed twice with PBS and lysed in ice-cold lysis buffer (1% Triton X-100, 50 mM Hepes pH 7.4, 150 mM NaCl, 1.5mM MgCl2, 1 mM EGTA, 100 mM NaF, 10 mM Sodium Pyrophosphate, 1 mM Na(subscript: 3)VO(subscript: 4), 10% glycerol, InmM phenylmethylsulfonyl fluoride, and 10 ug/ml aprotifin). Total protein concentration was determined using a BCA assay. 30ug of total cell lysate protein was diluted into Laemmli sample buffer and loaded onto a 10% acrylamide gel, subjected to SDS-PAGE, and transferred to a PVDF membrane. The membrane was blocked with 5% bovine serum albumin and then incubated at 4° C. overnight with antibody. The membrane was washed in TBS-T (10 mM Tris-HCl pH 7.4, 150 mM NaCl, and 0.1% Tween-20) and incubated with HRP-conjugated antibody (diluted in 5% milk in TBS-T) at room temperature for 1 h. The membrane was washed extensively and the proteins were visualized by chemiluminscent detection. The compounds effects on phospho-Akt levels were observed as relative differences in the amount of phospho-Akt detected by immunoblotting.

Effects on cellular levels of phospho-Akt following treatment with PI 3-K inhibitors were quantified using the PathScan phospho-Akt ELISA (Cell Signaling Technologies)., a sandwich ELISA for detection of phospho-Akt. The kit was used according to the manufacturer protocol. Absorbance at 450 nm was determined for each sample and used directly as equivalent of phospho-Akt levels.

Percent decreases in phospho-Akt levels were determined by normalizing relative to blank samples (0%/O) and control samples treated with DMSO alone (1 00%). Treatment with PI 3-K inhibitors resulted in a 20-60% decrease in phospho-Akt levels as determined by this assay. This data shows that these compounds are capable of affecting cellular PI 3-K mediated signaling.

Table 3 summarizes the data for several compounds of this structural group, including the IC₅₀ for inhibition of enzyme activity in vitro, cellular mIC₅₀ and anti-proliferative activity against tumor cells altered in PI 3-K mediated signaling, and effects on cellular levels of phospho-Akt.

TABLE 3
Summary of data for compounds of this invention

| | | | Anti-pro- | | | |
|---|---|---|---|---|---|---|
| | | | liferative | Approx. | | Approx. % |
| | | | effects on | mIC₅₀ | | decrease |
| | | | tumor cells | in | | phospho-Akt |
| | In vitro | | altered in | cellular | | relative to |
| Compound | IC₅₀ | | PI 3-K/PTEN | assays | | controls |
| 963985 | 500 | nM | Yes | 1 | μM | 50% |
| 964028 | 1 | μM | Yes | 10 | μM | 40% |
| 964076 | 3 | μM | Yes | 10 | μM | 60% |
| 964232 | 5 | μM | Yes | <20 | μM | 50% |
| 964247 | 7 | μM | Yes | <20 | μM | 30% |
| 964661 | 8 | μM | Yes | <20 | μM | 40% |
| 964260 | 8 | μM | Yes | <20 | μM | 40% |
| 963924 | <10 | μM | Yes | <20 | μM | |
| 964127 | <10 | μM | Yes | <20 | μM | |
| 964144 | <10 | μM | Yes | <20 | μM | |
| 964352 | <10 | μM | Yes | <20 | μM | |
| 964336 | <10 | μM | Yes | <20 | μM | |

### EXAMPLE 12

### Effects on tumor cells grown in 3-D culture systems by PI 3-K inhibitors.

PI 3-K inhibitors are assayed for effects on tumor cells grown in three-dimensional matrix that more closely mimics the environment of a tumor than other cell culture models. MA-MB-468 cells are mixed in a matrix solution, such as Matrigel (13D Biosciences) at 2×10 ⁶ cells/ml and 100 μl of this mixture added to each well of a 24 well cell culture plate. Each well is 6.5 mm in diameter and 2×10⁵ cells are added per well. Once the matrix is solidified, RMPI media containing 10% fetal calf serum and 2 mM L-glutamine is added to each well. After approximately 14 days of culture, the compounds are added to cell media at final concentrations ranging from 10 nM to 100 μM, and the cells are grown in the presence of the compounds for 7 days in RMPI media containing 0.5% fetal calf serum and 20 mM L-glutamine.

Following this treatment, cell growth in the three dimensional matrix can be measured using a cell viability assay such as the CellTiter 96 One Solution Cell Proliferation Assay (Promega, G3582). 1.2 ml of assay solution is added per well, the cells are incubated for 3 hours. Absorbance at 550 nm is determined for each well and used directly as being equivalent of cell number. In addition, live and dead cells can be distinguished and observed using fluorescence microscopy after staining with Fluorescein diacetate (Sigma), which labels live cells, and propidium iodide (Sigma), which labels dead cells.

The PI 3-K inhibitors of the present invention show anti-proliferative effects in this model of tumor cell growth, as shown by the representative data in Table 6, which compares the anti-proliferative effects of one inhibitor compared to the effects of the benchmark PI 3-K inhibitor LY294002. The PI 3-K inhibitors of the present invention also show enhanced anti-proliferative activity when combined with other cancer drugs, for example paclitaxel or doxorubicin.

TABLE 4
Effect of PI 3-K inhibitors in a three
dimensional model of tumor cell growth.

| | | |
|---|---|---|
| | | Percent Inhibition |
| Compound | Concentration | of Tumor Cell Viability |

| | | | |
|---|---|---|---|
| LY294002 | 50 | μM | 90% |
| | 10 | μM | 60% |
| | 5 | μM | 50% |
| | 1 | μM | <5% |
| CGX0963985 | 50 | μM | 40% |
| | 10 | μM | 30% |
| | 5 | μM | 20% |
| | 1 | μM | 10% |

### EXAMPLE 13

### Inhibition of Tumor Growth

The In vivo efficacy of an inhibitor of the growth of cancer cells may be confirmed by several protocols well known in the art. Human tumor cells which are deregulated in the PI 3-K pathway, for example, LnCaP, PC3, C33a, OVCAR-3, MDA-MB-468 are injected subcutaneously into the flank of nude mice on day 0. Mice are assigned to a vehicle, compound, or combination treatment group. Compound administration may begin on day 1-7. Subcutaneous administration may be done every day or every other day for the duration of the experiment, or the compound may be delivered by a continuous infusion pump.

The size of subcutaneous tumors can be monitored throughout the course of the experiment. The tumors are excised and weighed at the conclusion of the experiment and the average weight of tumors for each treatment group is calculated.

Alternatively, cell lines such as OVCAR-3 may be injected intraperitoneally into the abdominal cavity of female nude mice. Subcutaneous, intravenous, or intraperitoneal administration may be done every day or every other day for the duration of the experiment, or the compound may be delivered by a continuous infusion pump. The tumors are excised and weighed at the conclusion of the experiment and the average weight of the tumors for each treatment group is calculated. The PI 3-K inhibitors show enhanced activity against tumor growth when combined with other cancer drugs, for example paclitaxel or doxorubicin.

It is to be understood that the above-referenced arrangements are only illustrative of the application of the principles of the present invention. Numerous modifications and alternative arrangements can be devised without departing from the spirit and scope of the present invention. While the present invention has been shown in the drawings and is fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

1. A compound having a general structure represented by Formula I, Formula II, or Formula III; 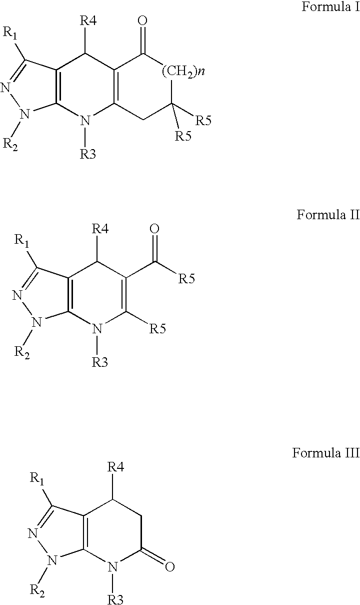
wherein n is an integer selected from 0 to 2;
R₁ and R₂ are each independently a member selected from the group consisting of hydrogen, alkyl, alkenyl, aryl, hetaryl, aralkyl, hetaralkyl, alkyl substituted with at least one substituent, aryl substituted with at least one substituent, hetaryl substituted with at least one substituent, aralkyl substituted with at least one substituent, and hetaralkyl substituted with at least one substituent;
R₃ is a member selected from the group consisting of hydrogen, alkyl, alkenyl, aralkyl, alkyl substituted with at least one substituent, aralkyl substituted with at least one substituent, CO—R₅, SO₂—R₅; CO—O—R₅, CO—N—R₄, and R₅; and
R₄ and R₅ are each independently a member selected from the group consisting of hydrogen, alkyl, alkenyl, cycloalkyl, aralkyl, aryl, alkyl substituted with at least one substituent, cycloalkyl substituted with at least one substituent, aryl substituted with at least one substituent, and aralkyl substituted with at least one substituent.

2. The compound according to claim 1, with reference to R₁₋₅, whenever the following are used;
alkyl is a straight or branched chain C₁₋₁₅ alkyl;
cycloalkyl is a C₃₋₈ cycloalkyl;
alkenyl is a straight or branched chain C₂₋₁₈ alkenyl;
aralkyl is a carbomonocyclic aromatic or carbobicyclic aromatic substituted with a straight or branched chain C₁₋₁₅ alkyl; and
substituent is selected from the group consisting of nitro, hydroxy, cyano, carbamoyl, mono- or di-C₁₋₄ alkyl-carbamoyl, carboxy, C₁₋₄ alkoxy-carbonyl, sulfo, halogen, C₁₋₄ alkoxy, phenoxy, halophenoxy, C₁₋₄ alkylthio, mercapto, phenylthio, pyridylthio, C₁₋₄ alkylsulfinyl, C₁₋₄ alkylsulfonyl, amino, C₁₋₃ alkanoylamino, mono- or di-C₁₋₄ alkylamino, 4- to 6-membered cyclic amino, C₁₋₃ alkanoyl, benzoyl, and 5 to 10 membered heterocyclic.

3. The compound according to claim 1, with reference to R₁₋₅, whenever the following are used;
aryl is a carbomonocyclic aromatic or carbobicyclic aromatic;
hetaryl is a heteromonocyclic aromatic or heterobicyclic aromatic containing 1 to 6 hetero-atoms selected from oxygen, sulfur and nitrogen;
aralkyl is a carbomonocyclic aromatic or carbobicyclic aromatic substituted with a straight or branched chain C₁₋₁₅ alkyl; and
substituent is a member selected from the group consisting of halogen, C₁₋₄ alkyl, C₁₋₄ haloalkyl, C₁₋₄ haloalkoxy, C₁₋₄ alkoxy, C₁₋₄ alkylthio, hydroxy, carboxy, cyano, nitro, amino, mono- or di-C₁₋₄ alkylamino, formyl, mercapto, C₁₋₄ alkyl-carbonyl, C₁₋₄ alkoxy-carbonyl, sulfo, C₁₋₄ alkylsulfonyl, carbamoyl, mono- or di-C₁₋₄ alkyl-carbamoyl, oxo, and thioxo.

4. The compound according to claim 1, wherein n is 1;
R₁ and R₂ are each independently a member selected from the group consisting of hydrogen, straight or branched chain C₁₋₆ alkyl, phenyl, naphthyl, hetaryl, C₁₋₆ alkyl substituted with at least one substituent, straight or branched chain C₁₋₆ alkylphenyl, phenyl substituted with at least one substituent, benzyl, and benzyl substituted with at least one substituent;
R₃ is a member selected from the group consisting of hydrogen, C₁₋₆ alkyl, aralkyl, C₁₋₆ alkyl substituted with at least one substituent, CO—R₅, or SO₂—R₅; CO—O—R₅, CO—N—R₄, and R₅;
R₄ and R₅ are each independently a member selected from the group consisting of hydrogen, C₁₋₆ alkyl, C₁₋₆ alkyl substituted with at least one substituent, cycloalkyl, phenyl, and phenyl substituted with at least one substituent, aralkyl, benzyl, and benzyl substituted with at least one substituent; and
substituent is a member selected from the group consisting of halogen, C₁₋₄ alkyl, C₁₋₄ haloalkyl, C₁₋₄ haloalkoxy, C₁₋₄ alkoxy, C₁₋₄ alkylthio, phenoxyl, halophenoxy, phenylthio, pyridylthio, hydroxy, carboxy, cyano, nitro, amino, C₁₋₃ alkanoylamino, mono- or di-C₁₋₄ alkylamino, 4- to 6-membered cyclic amino, formyl, mercapto, C₁₋₄ alkyl-carbonyl, C₁₋₄ alkoxy-carbonyl, sulfo, C₁₋₄ alkylsulfinyl, C₁₋₄ alkylsulfonyl, C₁₋₃ alkanoyl, benzoyl, mono- or di-C₁₋₄ alkyl-carbamoyl, oxo, thioxo, and 5 to 10 membered heterocyclic.

5. The compound according to claim 1, wherein n is 1, and with reference to R₁₋₅, whenever the following are used;
alkyl is a straight or branched chain C₁₋₁₅;
alkenyl is a straight or branched chain C₂₋₁₈;
aryl is a carbomonocyclic aromatic or carbobicyclic aromatic;
cycloalkyl is a C₃₋₈ alkyl ring,
hetaryl is a heteromonocyclic aromatic or heterobicyclic aromatic containing 1 to 6 hetero-atoms selected from the group consisting of oxygen, sulfur and nitrogen;
aralkyl is a carbomonocyclic aromatic or carbobicyclic aromatic and substituted with a straight or branched chain C-₁₋₁₅ alkyl;
hetaralkyl is a heteromonocyclic aromatic or heterobicyclic aromatic containing 1 to 6 hetero-atoms selected from the group consisting of oxygen, sulfur, and nitrogen and substituted with a straight or branched chain C₁₋₁₅ alkyl; and
substituent is a member selected from the group consisting of halogen, C₁₋₄ alkyl, C₁₋₄ haloalkyl, C₁₋₄ haloalkoxy, C₁₋₄ alkoxy, C₁₋₄ alkylthio, phenoxyl, halophenoxy, phenylthio, pyridylthio, hydroxy, carboxy, cyano, nitro, amino, C₁₋₃ alkanoylamino, mono- or di-C₁₋₄ alkylamino, 4- to 6-membered cyclic amino, formyl, mercapto, C₁₋₄ alkyl-carbonyl, C₁₋₄ alkoxy-carbonyl, sulfo, C₁₋₄ alkylsulfinyl, C₁₋₄ alkylsulfonyl, C₁₋₃ alkanoyl, benzoyl, mono- or di-C₁₋₄ alkyl-carbamoyl, oxo, thioxo, and 5 to 10 membered heterocyclic.

6. The compound according to claim 1, wherein n is 1;
R₁ and R₂ are each independently a member selected from the group consisting of straight or branched chain C_{1 6} alkyl, phenyl, benzyl, naphthyl, straight or branched chain C₁₋₆ alkyl substituted with at least one substituent, phenyl substituted with at least one substituent, and benzyl substituted with at least one substituent;
R₃ is a member selected from hydrogen, straight or branched chain C₁₋₆ alkyl, C₁₋₆ aralkyl, C₁₋₆ alkyl substituted with at least one substituent;
R₄ and R₅ are each independently a member selected from the group consisting of hydrogen, straight or branched chain C₁₋₆ alkyl, straight or branched chain C₁₋₆ alkyl substituted with at least one substituent, cycloalkyl, phenyl, phenyl substituted with at least one substituent, benzyl, and benzyl substituted with at least one substituent; and
substituent is a member selected from the group consisting of methyl, halogen, halophenyloxy, methoxy, ethyloxy phenoxy, benzyloxy, trifluromethyl, t-butyl, and nitro.

7. The compound according to claim 1, wherein n is 1;
R₁ is a member selected from the group consisting of straight or branched chain C₁₋₆ alkyl, and phenyl;
R₂ is a member selected from the group consisting of phenyl, C₁₋₆ alkylphenyl, C₁₋₆ dialkylphenyl, C₁₋₆ alkoxyphenyl, halophenyl, dihalophenyl, and nitrophenyl;
R₃ is a member selected from hydrogen and straight or branched chain C₁₋₆ alkyl;
R₄ is phenyl substituted with at least one substituent selected from the group consisting of halogen, phenoxy, benzyloxy, halophenoxy, straight or branched chain C₁₋₆ alkyl, C₁₋₆ alkoxy, and halo-C₁₋₄ alkyl and;
R₅ is a straight or branched chain C₁₋₆ alkyl.

8. The compound of claim 1, wherein n is 1;
R₁ is phenyl or t-butyl;
R₂ is a member selected from the group consisting of methylphenyl, dimethylphenyl, t-butyl, methoxyphenyl, chlorophenyl, dichlorophenyl, fluorophenyl, and nitrophenyl;
R₃ is hydrogen;
R₄ is a phenyl substituted with at least one substituent selected from the group consisting of chlorine, fluorine, phenoxy, benzyloxy, chlorophenoxy, methoxy, ethoxy, and trifluoromethyl; and
R₅ is a methyl.

9. The compound according to claim 1, wherein said compound has an IC₅₀ less than 10 μM in an in vitro inhibition of P I 3-K activity or an IC₅₀ less than 20 μM in cellular inhibition of P I 3-K activity.

10. A pharmaceutical composition comprising the compound or a salt thereof according to claim 1 and a pharmaceutically acceptable carrier.

11. A method of screening and characterizing the potency of a test compound as an inhibitor of phosphatidylinositol 3-kinase (PI 3-K) polypeptide, said method comprising the (a) measuring activity of a PI 3-K polypeptide in the presence of a test compound according to claim 1; and (b) comparing the activity of the PI 3-K polypeptide in the presence of the test compound to the activity of the PI 3-K polypeptide in the presence of an equivalent amount of a known PI 3-K inhibitor as a reference compound, wherein lower activity of the PI 3-K polypeptide in the presence of the test compound than in the presence of the reference compound indicates that the test compound is a more potent inhibitor than the reference compound, and higher activity of the PI 3-K polypeptide in the presence of the test compound than in the presence of the reference compound indicates that the test compound is a less potent inhibitor than the reference compound.

12. A method to treat a disorder in which P I 3-K plays a role, comprising administering to a patient with said disorder an effective amount of the compound or a salt thereof according to one of the claim 1.

13. The method according to claim 12, wherein the disorder is a cancer or a disease of immunity and inflammation.

14. The method according to claim 12, wherein the disorder is disruption of PI 3-K function in leukocytes.

15. A method for inhibiting growth of cancer cells, comprising contacting said cancer cells with an effective amount of the compound or a salt thereof according to claim 1.

16. The method according to claim 15, wherein said cancer cells are altered in PI 3-K mediated signaling via mutation in PTEN, amplification of the PIK3CA gene or mutations in PI 3-Kinase.

17. The method according to claim 15, wherein said cancers include breast, prostate, colon, lung, ovarian, and other cancers having altered PI 3-K activities.

18. A method for affecting PI 3-K mediated signaling in cells comprising contacting said cells with an effective amount of the compound or a salt thereof according to claim 1.

19. The method according to claim 18, wherein said compounds affect PI 3-K mediated phosphorylation of Akt.

20. A method for affecting PI 3-K mediated signaling in cells comprising contacting said cells with an effective amount of the compound or a salt thereof according to claim 2.
